SEQUENCE LISTING (32 sequences)

SEQ ID NO: 1 - DNA, 30 bp, Artificial Sequence
  Single strand DNA oligonucleotide
tcttctcgagatggcatcccccagacctct

SEQ ID NO: 2 - DNA, 30 bp, Artificial Sequence
  Single strand DNA oligonucleotide
aataagctttcagctggcggcgcaggtaag

SEQ ID NO: 3 - DNA, 31 bp, Artificial Sequence
  Single strand DNA oligonucleotide
tcttgtcgacatggccgtgacctccaacgag

SEQ ID NO: 4 - DNA, 29 bp, Artificial Sequence
  Single strand DNA oligonucleotide
aataagcttctagttggcgctgcaggaca

SEQ ID NO: 5 - DNA, 36 bp, Artificial Sequence
  Single strand DNA oligonucleotide
atatatccatggatggcatcccccagacctcttcgc

SEQ ID NO: 6 - DNA, 35 bp, Artificial Sequence
  Single strand DNA oligonucleotide
atatattctagatcactccaggctggcggcgcagg

SEQ ID NO: 7 - DNA, 38 bp, Artificial Sequence
  Single strand DNA oligonucleotide
tcagtctcatgaatggccgtgacctccaacgagaccgg

SEQ ID NO: 8 - DNA, 34 bp, Artificial Sequence
  Single strand DNA oligonucleotide
agcgtatctagactagttggcgctgcaggacacc

SEQ ID NO: 9 - DNA, 25 bp, Artificial Sequence
  Single strand DNA oligonucleotide
atttagctggctggcgtaaagtatg

SEQ ID NO: 10 - DNA, 25 bp, Artificial Sequence
  Single strand DNA oligonucleotide
tcatttcaggattgatcattgttgc

SEQ ID NO: 11 - DNA, 23 bp, Artificial Sequence
  Single strand DNA oligonucleotide
gacgaccccgaagacacaactag

SEQ ID NO: 12 - DNA, 1332 bp, Thermobifida fusca
atggcatcccccagacctcttcgcgctcttctgggcgccgcggcggcggccttggtcagcgcggctgctctggccttcccgtcgcaagcggcggccaatgattctccgttctacgtcaaccccaacatgtcctccgccgaatgggtgcggaacaaccccaacgacccgcgtaccccggtaatccgcgaccggatcgccagcgtgccgcagggcacctggttcgcccaccacaaccccgggcagatcaccggccaggtcgacgcgctcatgagcgccgcccaggccgccggcaagatcccgatcctggtcgtgtacaacgccccgggccgcgactgcggcaaccacagcagcggcggcgcccccagtcacagcgcctaccggtcctggatcgacgaattcgctgccggactgaagaaccgtcccgcctacatcatcgtcgaaccggacctgatctcgctgatgtcgagctgcatgcagcacgtccagcaggaagtcctggagacgatggcgtacgcgggcaaggccctcaaggccgggtcctcgcaggcgcggatctacttcgacgccggccactccgcgtggcactcgcccgcacagatggcttcctggctccagcaggccgacatctccaacagcgcgcacggtatcgccaccaacacctccaactaccggtggaccgctgacgaggtcgcctacgccaaggcggtgctctcggccatcggcaacccgtccctgcgcgcggtcatcgacaccagccgcaacggcaacggccccgccggtaacgagtggtgcgaccccagcggacgcgccatcggcacgcccagcaccaccaacaccggcgacccgatgatcgacgccttcctgtggatcaagctgccgggtgaggccgacggctgcatcgccggcgccggccagttcgtcccgcaggcggcctacgagatggcgatcgccgcgggcggcaccaaccccaacccgaaccccaacccgacgcccacccccactccgacccccacgccgcctcccggctcctcgggggcgtgcacggcgacgtacacgatcgccaacgagtggaacgacggcttccaggcgaccgtgacggtcaccgcgaaccagaacatcaccggctggaccgtgacgtggaccttcaccgacggccagaccatcaccaacgcctggaacgccgacgtgtccaccagcggctcctcggtgaccgcgcggaacgtcggccacaacggaacgctctcccagggagcctccacagagttcggcttcgtcggctctaagggcaactccaactctgttccgacccttacctgcgccgccagcctcgag

SEQ ID NO: 13 - protein, 444 aa, Thermobifida fusca
MASPRPLRALLGAAAAALVSAAALAFPSQAAANDSPFYVNPNMSSAEWVRNNPNDPRTPVIRDRIASVPQGTWFAHHNPGQITGQVDALMSAAQAAGKIPILVVYNAPGRDCGNHSSGGAPSHSAYRSWIDEFAAGLKNRPAYIIVEPDLISLMSSCMQHVQQEVLETMAYAGKALKAGSSQARIYFDAGHSAWHSPAQMASWLQQADISNSAHGIATNTSNYRWTADEVAYAKAVLSAIGNPSLRAVIDTSRNGNGPAGNEWCDPSGRAIGTPSTTNTGDPMIDAFLWIKLPGEADGCIAGAGQFVPQAAYEMAIAAGGTNPNPNPNPTPTPTPTPTPPPGSSGACTATYTIANEWNDGFQATVTVTANQNITGWTVTWTFTDGQTITNAWNADVSTSGSSVTARNVGHNGTLSQGASTEFGFVGSKGNSNSVPTLTCAASLE

SEQ ID NO: 14 - DNA, 891 bp, Thermobifida fusca
gccgtgacctccaacgagaccgggtaccacgacgggtacttctactcgttctggaccgacgcgcctggaacggtctccatggagctgggccctggcggaaactacagcacctcctggcggaacaccgggaacttcgtcgccggtaagggatgggccaccggtggccgccggaccgtgacctactccgccagcttcaacccgtcgggtaacgcctacctgaccctctacgggtggacgcggaacccgctcgtggagtactacatcgtcgaaagctggggcacctaccggcccaccggtacctacatgggcacggtgaccaccgacggtggtacctacgacatctacaagaccacgcggtacaacgcgccctccatcgaaggcacccggaccttcgaccagtactggagcgtccgccagtccaagcggaccagcggtaccatcaccgcggggaaccacttcgacgcgtgggcccgccacggtatgcacctcggaacccacgactacatgatcatggcgaccgagggctaccagagcagcggatcctccaacgtgacgttgggcaccagcggcggtggaaaccccggtgggggcaacccccccggtggcggcaacccccccggtggcggtggctgcacggcgacgctgtccgcgggccagcagtggaacgaccgctacaacctcaacgtcaacgtcagcggctccaacaactggaccgtgaccgtgaacgttccgtggccggcgaggatcatcgccacctggaacatccacgccagctacccggactcccagaccttggttgcccggcctaacggcaacggcaacaactggggcatgacgatcatgcacaacggcaactggacgtggcccacggtgtcctgcagcgccaactag

SEQ ID NO: 15 - protein, 296 aa, Thermobifida fusca
AVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNPGGGNPPGGGNPPGGGGCTATLSAGQQWNDRYNLNVNVSGSNNWTVTVNVPWPARIIATWNIHASYPDSQTLVARPNGNGNNWGMTIMHNGNWTWPTVSCSAN

SEQ ID NO: 16 - DNA, 106 bp, Lactobacillus plantarum
tatgaaaaaaattaataagttgatgatcttaggcatgctcgtttttggggtaacgggggcaacaatgatcaatcctgaaatgacgaccgcagcgcatgctagcgcc

SEQ ID NO: 17 - DNA, 124 bp, Lactobacillus plantarum
tatgaaaaaatttaactttaaaaccatgttgctattagttttggctagttgtgtcttcggggtcgtcgttaacgtgactactagtcttggaccacaaaccgcaatcaccgcccaggcctccaag

SEQ ID NO: 18 - DNA, 2094 bp, Clostridium clariflavum
atgaaacataaacttatgggaaaattttcgattttaatgagtcttacacttttgttatcactgttgccgttaaatgtgcaggcaaaaaccgttacctctaatgagaccggtactcatgggggctataactatgaatactggaaagacagcggcaacggaacaatggttctcaaggatggcggagctttcagctgcgaatggaataatattaacaatattttattccgcaagggtcttaaatttgatgagacaaagacccaccagcaaattggctatatgacactgacttattcgtgtgattatcaacctaacggtaattcatatctggctgtctatggatggacaagtgaccctcttgtagaatattacgttatcgagagctggggaacctggaagccgccaggaaatgtgcaatcaaaaggtactattaccgttgacggtggagtatacgacatatttgaaacaaccagatacaatcagccttccataaaaggtaatgcaacattccagcaatattggagtgttcgccagcagaaacgtacaagcggaacagtatctgttactgaacactttaaagcttgggaagctaaaggaatgaagatgggaaaattctatgaaatttcccttgttgtagaaggatatcaaagcagcggtaaagccgatgtaaccatgatgtcaattaatattggcggaaattcagcaaatcctaccggtaccactgttgtgccaactcctgcatcgggttccgggaaaagtgctttttcaactatagaagccgaagactttgacaatgcttacggttcaagcataagatccattggtatgggtgtaggttacatagaaaacggtaactacctggtatataacaatatcaattttggtgacggagcaagtgcttttagtgccaaagttgctaacggtaatactactgcaacaactattcaattaagattaggcagcccgagcggtactctgataggttctttaagtgtgccctcaaccggtggctggaacaattatgaagaattatccactaccgtaagcggagcatccggaacaaaagacctgtacctctgctttaacggacctgtaaatattgattgtttctcctttgcgaaaggaaattccaatacaaatcctataaatccgggctacatattgcttggtgacgttgaccagaacggtactataaattctttggattatgccaaatacaaaatgtatttgcttggaatgataagttctcttcctgaggaaggggatataaacagagacggcgacatgaactcaattgactatgcaatgctgaaacagcatcttttgggtataattaatttggaagagctttcaggacctaaaacaacacctacaccggttgtgattgttacaccgactcctacaaaaacgccatctgaaggtacattccactgcttcttgctgcttggtcagtcgaatatggcaggatgggccagggctcaagattctgacaagatacctaatccgcgcatacttgcactgggctatgacaataatcagtggggtgtagctgtaccgccgttgcatgaagcatttcaaggcgctataggtcctggtgactggtttgctaaaacaataattgaaagacttcctgaaaatgatactataggattgataccgtgtgctataagcggtgaaaagatcgaaaccttcatgaaaaatggaggatcaaaatataattggattgtcagccgtgcaagaatggcacagcaaagaggaggagttattgaaggtatcctcttccaccagggtgagtccaacaacggtcagcaagattggcctaataaagtaagtacactgatttcagacctcaaaaaagacttagggcttggagatattcctgttctggtgggagaattgctctataccggtagctgtgcagggcataatactttggtaaacagactgccgtcaatgattccgaattgttatgttatttctgcacaaggtttatccggtgacccggcagatttctggggactccatttcaatcatgattctactgttgaattcggtaagagatatgcgaaaaaaatgattgaagtacttggatggtaa

SEQ ID NO: 19 - protein, 697 aa, Clostridium clariflavum
MKHKLMGKFSILMSLTLLLSLLPLNVQAKTVTSNETGTHGGYNYEYWKDSGNGTMVLKDGGAFSCEWNNINNILFRKGLKFDETKTHQQIGYMTLTYSCDYQPNGNSYLAVYGWTSDPLVEYYVIESWGTWKPPGNVQSKGTITVDGGVYDIFETTRYNQPSIKGNATFQQYWSVRQQKRTSGTVSVTEHFKAWEAKGMKMGKFYEISLVVEGYQSSGKADVTMMSINIGGNSANPTGTTVVPTPASGSGKSAFSTIEAEDFDNAYGSSIRSIGMGVGYIENGNYLVYNNINFGDGASAFSAKVANGNTTATTIQLRLGSPSGTLIGSLSVPSTGGWNNYEELSTTVSGASGTKDLYLCFNGPVNIDCFSFAKGNSNTNPINPGYILLGDVDQNGTINSLDYAKYKMYLLGMISSLPEEGDINRDGDMNSIDYAMLKQHLLGIINLEELSGPKTTPTPVVIVTPTPTKTPSEGTFHCFLLLGQSNMAGWARAQDSDKIPNPRILALGYDNNQWGVAVPPLHEAFQGAIGPGDWFAKTIIERLPENDTIGLIPCAISGEKIETFMKNGGSKYNWIVSRARMAQQRGGVIEGILFHQGESNNGQQDWPNKVSTLISDLKKDLGLGDIPVLVGELLYTGSCAGHNTLVNRLPSMIPNCYVISAQGLSGDPADFWGLHFNHDSTVEFGKRYAKKMIEVLGW

SEQ ID NO: 20 - DNA, 1374 bp, Clostridium thermocellum
atgaaacaaaaattactggtaactttcctgattttaattacttttaccgtttcgctgactttgtttccggtaaatgtacgcgctgatgtggtaattacgtcaaaccagacgggtactcacggcgggtacaactttgagtactggaaagacaccggaaacggaaccatggtcctcaaagacggtggtgcgttcagctgcgaatggagcaatatcaacaatattcttttccgtaaaggtttcaaatacgatgaaacaaagacacatgatcaacttggatacataacggtaacttattcctgcaactatcagccaaacggaaactcttatctgggagtctacggatggaccagcaatccgcttgtagagtattacatcatcgagagctggggaacctggagaccaccgggagcaacaccaaagggcactattaccgttgacggtggtacatacgagatatacgagaccaccagagttaaccagccttccatcaaaggtacagctactttccagcaatactggagtgtacgtacatcaaaacgtacaagcggaaccatatccgtaaccgaacactttaaagcctgggaacgtctgggtatgaaaatgggaaaaatgtatgaggttgctttggttgtagaaggataccagagcagcggaaaagccgacgtaaccagcatgacaattactgttggcaacgcaccgtcaacatcatcaccaccgggtccgacacctgaaccgactccaagaagtgctttttcaaaaatcgaagctgaggagtacaactccctcaagtcatcaaccattcagaccataggcacttccgacggaggaagcggtataggttatattgaaagcggtgactatctggtatttaacaaaataaactttggaaacggtgcaaactctttcaaggcaagggttgcatccggtgcggacacacccaccaatatccagttaagactcggaagcccgaccggtactcttataggaactcttacggtggcttccacaggcggttggaacaattacgaggaaaaatcctgcagcataaccaacactacaggacagcacgacttatatctggtattctcaggtcctgttaacattgactacttcatattcgactcgaaaggtgtaaatcctacacctacacctacttctccgcctcaacaagaccaggttttgggtgacttgaacggagacaaacaagtaaattcaacagactatacagcactgaagagacatttgctcaatataaccagactttcaggaaccgctcttgccaatgccgatgtaaaccgcgacggcaaagttgattccactgatcttatgatgttgcacagataccttcttcgtataatctccaaacttggatga

SEQ ID NO: 21 - protein, 457 aa, Clostridium thermocellum
MKQKLLVTFLILITFTVSLTLFPVNVRADVVITSNQTGTHGGYNFEYWKDTGNGTMVLKDGGAFSCEWSNINNILFRKGFKYDETKTHDQLGYITVTYSCNYQPNGNSYLGVYGWTSNPLVEYYIIESWGTWRPPGATPKGTITVDGGTYEIYETTRVNQPSIKGTATFQQYWSVRTSKRTSGTISVTEHFKAWERLGMKMGKMYEVALVVEGYQSSGKADVTSMTITVGNAPSTSSPPGPTPEPTPRSAFSKIEAEEYNSLKSSTIQTIGTSDGGSGIGYIESGDYLVFNKINFGNGANSFKARVASGADTPTNIQLRLGSPTGTLIGTLTVASTGGWNNYEEKSCSITNTTGQHDLYLVFSGPVNIDYFIFDSKGVNPTPTPTSPPQQDQVLGDLNGDKQVNSTDYTALKRHLLNITRLSGTALANADVNRDGKVDSTDLMMLHRYLLRIISKLG

SEQ ID NO: 22 - DNA, 1008 bp, Thermobifida halotolerans
atgaacgacgcccccgcccacccaaagagccgcagacacggccgcatcagactgttcgtcggccgtgtctgcaccgccctggtggcgctggtcacggccacgacgatgctgcccggcgtcgccaacgcggccgtgacctccaaccagacgggcacccatgacggctatttctactcgttctggaccgacagccccggaacggtctccatggagctgggccccggaggcaactacagcacctcgtggagcaacaccgggaacttcgttgtcggtaagggctggagcaccggcggacgcaggaccgtgacctactccggtagcttcaacccgtccggtaacgcgtacctgactctctacgggtggacgagaaacccgctcgtggagtactacatcgtcgacaactggggcacctaccggcccacgggaacgtacaagggcacggtcaccagcgacggcggcacgtacgacatctacgagacgacgcgcaccaacgccccctccatcgagggcaccgcaaccttcaagcagtactggagcgtccggcagtcgaggagaacgggcggaaccataaccgctggaaaccacttcgacgcatgggcccgccacggaatgaacctgggcagccatgactacatgatcatggcgacggagggataccagagcagcgggagctccaacataacggtgggcggctccggcggcggcaatcctggcggaaaccccggcggcaaccccggtggcggtggctgcaccgcgaccctgtccgcggggcagcagtggagtgaccggtacaacctcggcgtctcggtcagcggctccagcaactggaccgtgacgatgaacgtgccgtccccggcgaagatcatcgccacgtggaacatcagcgccagctaccccaacgcccagacgctgaccgccaggcccaacggcaacggcaacaactggggtgtgacgatccagcacaacggcaactggacctggccgacggtctcctgcagcgcgaactga

SEQ ID NO: 23 - protein, 335 aa, Thermobifida halotolerans
MNDAPAHPKSRRHGRIRLFVGRVCTALVALVTATTMLPGVANAAVTSNQTGTHDGYFYSFWTDSPGTVSMELGPGGNYSTSWSNTGNFVVGKGWSTGGRRTVTYSGSFNPSGNAYLTLYGWTRNPLVEYYIVDNWGTYRPTGTYKGTVTSDGGTYDIYETTRTNAPSIEGTATFKQYWSVRQSRRTGGTITAGNHFDAWARHGMNLGSHDYMIMATEGYQSSGSSNITVGGSGGGNPGGNPGGNPGGGGCTATLSAGQQWSDRYNLGVSVSGSSNWTVTMNVPSPAKIIATWNISASYPNAQTLTARPNGNGNNWGVTIQHNGNWTWPTVSCSAN

SEQ ID NO: 24 - DNA, 1005 bp, Thermobispora bispora
atgttcaggtccccgagaggccacgggcgcggccgtaggttcatcggccggatctgcgccttagccgtggcggtgatcaccgggctgacgatgtcggtcggcaccgcccacgcccagacgatcagctcgaaccagaccggctaccacgacggctacttctactccttctggaccgatgcccccggcacggtctcgatgaccctcggatcgggcgggaactacagcacctcgtggtggaacaccgggaacttcgtcgcgggtaagggctgggccaccggcggccgccggacggtgacctactcggccagcttcaacccgtccggcaacgcctacctggcgctgtacgggtggacccgtaacccgctcgtcgagtactacatcgtcgagagctggggcacctaccggccgacgggcacctacaagggcaccgtcaccaccgacggcggcacctacgacatctacgagacgacccggtacaacgccccctccatcgagggcacccggaccttcaagcagttctggagcgtccggcagtcgaagcggaccagcggcaccatcaccaccggcaaccacttcgacgcctgggcccgcgcggggatgcagctcggcagcttcgactacatgatcatggccaccgagggctaccagagcagcggcaactccaacgtcaccctgggcaccggcggcggtggcggcaccacgcctccgcccggtggcggcggtggcggtggcggcggtggctgccaggccacgatcatcccgggccaggtgtgggccgaccggttcaacctgtcggtccaggtcaccggcaccgacaactgggtcgtcacggtcaccgtgaccccgccgcagaagatcatcgccacctggaacggcaatccgacctgggattacacaggcaacgtgatgaccatgaggcccaacggcagcgggaacacgttcggtttcacgatcatgcacaacggcaactggagctggccgagcgtgacctgccgggtggcctga

SEQ ID NO: 25 - protein, 334 aa, Thermobispora bispora
MFRSPRGHGRGRRFIGRICALAVAVITGLTMSVGTAHAQTISSNQTGYHDGYFYSFWTDAPGTVSMTLGSGGNYSTSWWNTGNFVAGKGWATGGRRTVTYSASFNPSGNAYLALYGWTRNPLVEYYIVESWGTYRPTGTYKGTVTTDGGTYDIYETTRYNAPSIEGTRTFKQFWSVRQSKRTSGTITTGNHFDAWARAGMQLGSFDYMIMATEGYQSSGNSNVTLGTGGGGGTTPPPGGGGGGGGGGCQATIIPGQVWADRFNLSVQVTGTDNWVVTVTVTPPQKIIATWNGNPTWDYTGNVMTMRPNGSGNTFGFTIMHNGNWSWPSVTCRVA

SEQ ID NO: 26 - DNA, 1035 bp, Thermopolyspora flexuosa
atgaacgaacccctcaccatcacgcaggccaggcgccgcagacgcctcggcctccggcgcatcgtcaccagtgccttcgccctggcactcgccatcgccggtgcgctgctgcccggcacggcccacgccgacaccaccatcacccagaaccagaccgggtacgacaacggctacttctactcgttctggaccgacgcgcccgggaccgtctccatgaccctccactcgggcggcagctacagcacctcgtggcggaacaccgggaacttcgtcgccggcaagggctggtccaccggcggacggcggaccgtgacctacaacgcctccttcaacccgtcgggtaacgcctacctcacgctctacggctggaccaggaacccgctcgtcgagtactacatcgtcgagagctggggcacctaccggcccaccggcacctacaagggcaccgtcaccaccgacggcggcacgtacgacatctacgagacctggcggtacaacgcgccgtccatcgagggcacccggaccttccagcagttctggagcgtccggcagcagaagcggaccagcggcaccatcaccatcggcaaccacttcgacgcctgggcccgcgccggcatgaacctgggcagccacgactaccagatcatggcgaccgagggctaccagagcagcggtagctccaccgtctccatcagcgagggtggcaaccccggcaacccgggtaaccccggcaaccccggcaaccccggtaacccgggtaaccccggcggtggctgcgtcgcgaccctctccgccggccagcagtggagcgaccgctacaacctcaacgtctcggtcagcggctcgaacaactggacggtccggatggacgtgccctacccggcccgcatcatcgccacctggaacatccacgcccagtggcccgagtcccaggtgctcatcgccagacccaacggcaacggcaacaactggggcgtgacgatccagcacaacggcaactggacctggccgacggtcacctgtaccgcgaactga

SEQ ID NO: 27 - protein, 344 aa, Thermopolyspora flexuosa
MNEPLTITQARRRRRLGLRRIVTSAFALALAIAGALLPGTAHADTTITQNQTGYDNGYFYSFWTDAPGTVSMTLHSGGSYSTSWRNTGNFVAGKGWSTGGRRTVTYNASFNPSGNAYLTLYGWTRNPLVEYYIVESWGTYRPTGTYKGTVTTDGGTYDIYETWRYNAPSIEGTRTFQQFWSVRQQKRTSGTITIGNHFDAWARAGMNLGSHDYQIMATEGYQSSGSSTVSISEGGNPGNPGNPGNPGNPGNPGNPGGGCVATLSAGQQWSDRYNLNVSVSGSNNWTVRMDVPYPARIIATWNIHAQWPESQVLIARPNGNGNNWGVTIQHNGNWTWPTVTCTAN

SEQ ID NO: 28 - protein, 1228 aa, Acidothermus cellulolyticus
MERTQQSGRNCRYQRGTTRMPAISKRLRAGVLAGAVSIAASIVPLAMQHPAIAATHVDNPYAGATFFVNPYWAQEVQSEAANQTNATLAAKMRVVSTYSTAVWMDRIAAINGVNGGPGLTTYLDAALSQQQGTTPEVIEIVIYDLPGRDCAALASNGELPATAAGLQTYETQYIDPIASILSNPKYSSLRIVTIIEPDSLPNAVTNMSIQACATAVPYYEQGIEYALTKLHAIPNVYIYMDAAHSGWLGWPNNASGYVQEVQKVLNASIGVNGIDGFVTNTANYTPLKEPFMTATQQVGGQPVESANFYQWNPDIDEADYAVDLYSRLVAAGFPSSIGMLIDTLRNGWGGPNEPTGPSTATDVNTFVNQSKIDLRQHRGLWCNQNGAGLGQPPQASPTDFPNAHLDAYVWIKPPGESDGTSAASDPTTGKKSDPMCDPTYTTSYGVLTNALPNSPIAGQWFPAQFDQLVANARPAVPTSTSSSPPPPPPSPSASPSPSPSPSPSSSPSPSPSPSSSPSPSPSPSPSPSSSPSPSPSSSPSPSPSPSPSPSSSPSPSPSSSPSPSPSPSPSPSSSPSPSPTSSPVSGGLKVQYKNNDSAPGDNQIKPGLQLVNTGSSSVDLSTVTVRYWFTRDGGSSTLVYNCDWAAMGCGNIRASFGSVNPATPTADTYLQLSFTGGTLAAGGSTGEIQNRVNKSDWSNFTETNDYSYGTNTTFQDWTKVTVYVNGVLVWGTEPSGTSPSPTPSPSPSPSPSPGGDVTPPSVPTGLVVTGVSGSSVSLAWNASTDNVGVAHYNVYRNGVLVGQPTVTSFTDTGLAAGTAYTYTVAAVDAAGNTSAPSTPVTATTTSPSPSPTPTGTTVTDCTPGPNQNGVTSVQGDEYRVQTNEWNSSAQQCLTINTATGAWTVSTANFSGGTGGAPATYPSIYKGCHWGNCTTKNVGMPIQISQIGSAVTSWSTTQVSSGAYDVAYDIWTNSTPTTTGQPNGTEIMIWLNSRGGVQPFGSQTATGVTVAGHTWNVWQGQQTSWKIISYVLTPGATSISNLDLKAIFADAAARGSLNTSDYLLDVEAGFEIWQGGQGLGSNSFSVSVTSGTSSPTPSPSPTPTPSPTPTPSPSPTPSPSPTSSPSSSGVACRATYVVNSDWGSGFTATVTVTNTGSRATNGWTVAWSFGGNQTVTNYWNTALTQSGASVTATNLSYNNVIQPGQSTTFGFNGSYSGTNAAPTLSCTAS

SEQ ID NO: 29 - DNA, 1371 bp, Thermobispora bispora
atgtctcgaataaggcgctttttggccacagcgctcgccgcggccaccgccggcgtgggggcgatcgtcaccgccatcgcctcggccggccccgctcacgcctatgactcgcccttctacgtggacccgcagagcaacgcggccaagtgggtggcggccaaccccaacgacccccggacgccggtgatccgggaccggatcgccgcggtgccgaccggccgctggttcgccaactacaacccctcgaccgtgcgcgccgaggtcgacgcgtacgtcggcgccgcggccgcggccgggaagatcccgatcatggtcgtgtacgccatgccgaaccgggactgcggcgggccgagcgcgggcggtgcccccaaccacaccgcctaccgcgcctggatcgatgagatcgccgccggcctccgcaaccgcccggccgtgatcatcctggagccggacgcgctcccgatcatgaccaactgcatgagccccagcgagcaggccgaggtccaggcctccatggcgtacgcgggcaagaagttcaaggccgcctcgtcgcaggcgaaggtctacttcgacgccggccacgacgcctgggtcccggcggacgagatggcctcccgcctcaggggcgccgacatcgccaacagcgcggacggcatcgcgctcaacgtctccaactaccggtacacctcgggcctgatctcgtacgccaagagcgtcctctcggcgatcggcgcgtcgcacctgcgcgcggtcatcgacaccagccggaacggcaacggcccgctcggcagcgagtggtgcgacccgccgggccgcgccaccggcacctggagcaccacggacaccggcgacccggcgatcgacgccttcctgtggatcaagccgccgggtgaggccgacgggtgcatcgccacgccgggtgtcttcgtcccggaccgggcctacgagctcgccatgaacgcggcgccgcccacgtacagcccgtcgcccacgccgagcagcccctccccctcgccgtcccagagcgacccgggttcgccctcgccgtcgccgtcccagccgccggcgggcaaggcgtgcgaggccacgtacgcgctggtcaaccagtggccgggcggcttccaggccgaggtgaccgtgaagaacaccggctcctcgccgatcaacgggtggaccgttcagtggaccctgccgagcggccagtccatcacccagctgtggaacggtgacctgtccaccagcggctcgaacgtcaccgtccggaacgtgagctggaacggcaacgtgccggccggcggctccacctcgttcggcttcctcggctcgggcaccggccagctctcctcctccatcacctgctcggcgagctga

SEQ ID NO: 30 - protein, 456 aa, Thermobispora bispora
MSRIRRFLATALAAATAGVGAIVTAIASAGPAHAYDSPFYVDPQSNAAKWVAANPNDPRTPVIRDRIAAVPTGRWFANYNPSTVRAEVDAYVGAAAAAGKIPIMVVYAMPNRDCGGPSAGGAPNHTAYRAWIDEIAAGLRNRPAVIILEPDALPIMTNCMSPSEQAEVQASMAYAGKKFKAASSQAKVYFDAGHDAWVPADEMASRLRGADIANSADGIALNVSNYRYTSGLISYAKSVLSAIGASHLRAVIDTSRNGNGPLGSEWCDPPGRATGTWSTTDTGDPAIDAFLWIKPPGEADGCIATPGVFVPDRAYELAMNAAPPTYSPSPTPSSPSPSPSQSDPGSPSPSPSQPPAGKACEATYALVNQWPGGFQAEVTVKNTGSSPINGWTVQWTLPSGQSITQLWNGDLSTSGSNVTVRNVSWNGNVPAGGSTSFGFLGSGTGQLSSSITCSAS

SEQ ID NO: 31 - DNA, 1260 bp, Themomonospora curvata
atggcaccccctcgcggcgccccggcccccgtgcgcgcccggctgcgcgcctggtcggcccgctgcgccgcgctcgcgaccgcggcggccctcaccgccgcagcggcgccgcccgcccaggcgcatgccgccggcaaccccctgcagggcccgagggcggcgaaggtccgcttcttcgtcgaacccgacaccaacgccggacggcaggcccgggtgtgggccgcgcagggacgcttccacgacgccgccctcatgcgggcgctgtcgaagatctcccaggcggtctggttcaccgagggaactccccagcaggtgcagcggcgggtccgcgagaccatgcgccaggcccgccgccaaggcgccgtccccgtgctggtggcctactacgtgccgggacgggactgctcgcagtactcggccggcggcgcccccagcgagcgggcctaccgggagtggatcaacgccttcgcccgcggcatcggcggcggccgggccgtggtgatcgtcgaacccgacgggctggccctgctgtccagcgagccgtggtgcaacgaaggcggcggcggctccaccggccggccggaggacatgtcgctggtcgagcagcgcttccgggagatcgaccacgccatcaccaccttcgccaagctccccaacaccggcgtgtacgtggacgccggccactcggcctggcagccgctcaacgactacgacgccggctacggcgagccgcgcgcccagctcggcatcgtcagccgcctgctgcgcggcggcgtcgccaaggccgacgggttcgtgctgaacgtctccaactaccgggccgacgccgagctgatcgactacggcgtccgggtctccaagtgcctgtggctgcgccgcagcaccggcgcgcgcgagtgcaccgacgccgacctggccgccgtgcccgacggccggcgcgacctgacccccttcgtcctggacaccagccgcaacggccgggggccgtggacggcgccggagggcgcgtaccccgacccccaggagtggtgcaacccgcccggccgcggcctgggcgtccgccccaccacccgcaccggccaccgcctggtggacgccttcctgtgggtcaaacggcccggcgagtccgacggccagtgcacccgcggcaccgccggaccgcaggaccccgagtacggcatcgtcgacccgcccgcgggccagtggtggcccgagtacgccctgggcctggcccggcgggccgtgccacccctcaagtga

SEQ ID NO: 32 - protein, 419 aa, Themomonospora curvata
MAPPRGAPAPVRARLRAWSARCAALATAAALTAAAAPPAQAHAAGNPLQGPRAAKVRFFVEPDTNAGRQARVWAAQGRFHDAALMRALSKISQAVWFTEGTPQQVQRRVRETMRQARRQGAVPVLVAYYVPGRDCSQYSAGGAPSERAYREWINAFARGIGGGRAVVIVEPDGLALLSSEPWCNEGGGGSTGRPEDMSLVEQRFREIDHAITTFAKLPNTGVYVDAGHSAWQPLNDYDAGYGEPRAQLGIVSRLLRGGVAKADGFVLNVSNYRADAELIDYGVRVSKCLWLRRSTGARECTDADLAAVPDGRRDLTPFVLDTSRNGRGPWTAPEGAYPDPQEWCNPPGRGLGVRPTTRTGHRLVDAFLWVKRPGESDGQCTRGTAGPQDPEYGIVDPPAGQWWPEYALGLARRAVPPLK

(19) United States
(12) Patent Application Publication
(10) Pub. No.: US 2016/0326485 A1
(43) Pub. Date: Nov. 10, 2016

(54) LACTIC ACID BACTERIA GENETICALLY MODIFIED TO EXPRESS ENZYMES OF THE CELLULOLYTIC SYSTEM

(71) Applicants: The State of Israel, Ministry of Agriculture & Rural Developmemt, Agricultural Research, Rishon-LeZion (IL); Yeda Research and Development Co. Ltd., Rehovot (IL); Ramot at Tel-Aviv University Ltd., Tel-Eviv (IL)

(72) Inventors: Itzhak MIZRAHI, Tel-Aviv (IL); Naama SHTERZER, Tel-Aviv (IL); Raphael LAMED, Yavne (IL); Inna ROZMAN GRINBERG, RaAnana (IL); Edward A. BAYER, Ramot-HaShavim (IL); Sarah MORAIS, Ashdod (IL)

(21) Appl. No.: 15/108,410

(22) PCT Filed: Dec. 26, 2013
(86) PCT No.: PCT/IL2013/051071

§ 371 Date: Jun. 27, 2016

(51) Int. Cl. C12N 1/22; C12N 9/24; C12N 1/20

(52) U.S. Cl. CPC C12N 1/22; C12N 1/20; C12N 9/2482; C12Y 302/01008

(57) ABSTRACT

Lactic acid cell cultures for processing lignocellulose are disclosed. The bacterial culture may comprise a biomass composition and a population of lactic acid bacteria which comprises:

(i) a first population of lactic acid bacteria which has been genetically modified to express a secreted cellulase; and

(ii) a second population of lactic acid bacteria which has been genetically modified to express a secreted xylanase, wherein the ratio of the first population: second population is selected such that the specific activity of cellulase:xylanase in the culture is greater than 4:1 or less than 1:4.

### FIELD AND BACKGROUND OF THE INVENTION

The present invention, in some embodiments thereof, relates to lactic acid bacteria genetically modified to express enzymes of the cellulotyic system and, more particularly, but not exclusively, to a cellulase and a xylanase.

Plant cell wall fibers are composed of polymeric components such as cellulose, lignin, pectins and hemicelluloses, that collectively represent the most abundant renewable organic polymers on Earth. Despite its recalcitrant nature, the polysaccharides of the plant cell wall provide an exceptional source of carbon and energy, and a multitude of different microorganisms have evolved enzyme systems (notably glycoside hydrolases), which are capable of degrading plant cell wall polysaccharides. Exploiting these enzymes in biotechnological process, e.g. via metabolic engineering, holds great environmental and applicative potential.

One attractive candidate for metabolic engineering towards plant mass bioprocessing is Lactobacillus plantarum, which is a common lactic acid bacterium with homolactic fermentation on hexose sugars and heterolactic (lactic+acetic acid) fermentation on pentoses. L plantarum is used in a variety of industrial and agricultural applications and prospers in environments containing decomposed lignocellulosic plant biomass (2). In agriculture, the acidifying properties of these organisms are employed for conservation of plant biomass for use in animal feed (3). The ability to produce lactic acid in large amounts could also be used for the production of biobased plastics (polylactic acid) from plant biomass. Interestingly, Lactobacillus species are predominant in contaminated ethanol fermentations (4, 5) and L. plantarum shows high ethanol tolerance (6), rendering it as a possible candidate for production of biofuel by introduction of ethanol-producing enzymes into its genetic repertoire (7). In contrast to the commonly used ethanol producing yeast Saccharomyces cerevisiae, L. plantarum is able to metabolize pentose sugars derived from lignocellulosic biomass (8-11). The production of acid and the bacterium's acid tolerance reduces the risk of contamination by other bacteria and fungi and may enable degradation of substrates directly after acid pretreatments that are commonly used for lignin deconstruction in plant biomass. L. plantarum contains 55 genes encoding for 18 glycosides hydrolases families but none are strict cellulases or xylanases (12). Consequently the bacterium lacks the inherent ability to degrade cellulose and hemicelluloses.

Expression of cellulases from Gram-positive bacteria in L. plantarum, using heterologous promoters and secretion signals has been reported (12-14). Intracellular expression with the pSIP system has recently been used for the expression of a recombinant Pyrococcus furiosus cellulase in both L. plantarum and L. casei strains (19).

Additional background art includes Ozkose et al., Folia microbiologica 54:335-342; Liu et al., Applied microbiology and biotechnology 77:117-124; Bates et al., 1989 Appl. Environ. Microbiol. 1989, 55(8):2095; Scheirlinck et al., 1989, Appl. Environ. Microbiol. 1989, 55(9):2130; Scheirlinck et al., 1990, AppI Microbiol Biotechnol., 33:534-541; Rossi et al., Antonie van Leeuwenhoek 80: 139-147, 2001.

### SUMMARY OF THE INVENTION

According to an aspect of some embodiments of the present invention there is provided a bacterial culture comprising a biomass composition and a population of lactic acid bacteria which comprises:

(i) a first population of lactic acid bacteria which has been genetically modified to express a secreted cellulase; and

(ii) a second population of lactic acid bacteria which has been genetically modified to express a secreted xylanase, wherein the ratio of the first population: second population is selected such that the specific activity of cellulase:xylanase in the culture is greater than 4:1 or less than 1:4.

According to an aspect of some embodiments of the present invention there is provided a population of lactic acid bacteria which have been genetically modified to express a secreted cellulase and a secreted xylanase.

According to an aspect of some embodiments of the present invention there is provided a culture comprising the population of lactic acid bacteria described herein and a biomass composition.

According to an aspect of some embodiments of the present invention there is provided silage comprising the culture described herein.

According to an aspect of some embodiments of the present invention there is provided a silage generated by processing lignocelluloses according to the method described herein.

According to an aspect of some embodiments of the present invention there is provided a method of processing lignocellulose comprising propagating the culture described herein, thereby processing the lignocellulose.

According to some embodiments of the invention, the molar ratio of cellulase:xylanase in the culture is greater than 4:1 or less than 1:4.

According to some embodiments of the invention, the molar ratio of cellulase:xylanase in the culture is greater than 10:1 or less than 1:10.

According to some embodiments of the invention, the molar ratio of cellulase:xylanase in the culture is less than 1:10.

According to some embodiments of the invention, the expression plasmid for expressing the secreted cellulase and the secreted xylanase is a pSIP-derived expression plasmid.

According to some embodiments of the invention, the biomass comprises lignocelluloses.

According to some embodiments of the invention, the biomass comprises cellulose and hemicellulose.

According to some embodiments of the invention, the biomass further comprises lignin.

According to some embodiments of the invention, the biomass is selected from the group consisting of paper, paper products, paper waste, wood, particle board, sawdust, agricultural waste, sewage, silage, grasses, rice hulls, bagasse, jute, hemp, flax, bamboo, sisal, abaca, straw, corn cobs, corn stover, switchgrass, alfalfa, hay, coconut hair, cotton, seaweed, algae, and mixtures thereof.

According to some embodiments of the invention, the lactic acid bacteria are Lactobacillus plantarum.

According to some embodiments of the invention, the first population of lactic acid bacteria comprises Lactobacillus plantarum.

According to some embodiments of the invention, the second population of lactic acid bacteria comprises Lactobacillus plantarum.

According to some embodiments of the invention, the cellulase is a Thermobifida fusca cellulase.

According to some embodiments of the invention, the xylanase is a Thermobifida fusca xylanase.

According to some embodiments of the invention, the cellulase is a mesophilic bacteria cellulase.

According to some embodiments of the invention, the xylanase is a mesophilic bacteria xylanase.

According to some embodiments of the invention, the mesophilic bacteria is a Ruminococcus flavefaciens or Ruminococcus albus.

According to some embodiments of the invention, the cellulase comprises a leader sequence as set forth in SEQ ID NOs: 16 or 17.

According to some embodiments of the invention, the xylanase comprises a leader sequence as set forth in SEQ ID NOs: 16 or 17.

According to some embodiments of the invention, the first population and the second population comprise identical strains of bacteria.

According to some embodiments of the invention, the first population and the second population comprise non-identical strains of bacteria.

According to some embodiments of the invention, the method further comprises degrading the lignin of the lignocellulose.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are bar graphs illustrating the comparative enzymatic activity of purified recombinant Cel6A (A) and Xyn11A (B) enzymes on PASC or xylan, at 37° C. or 50° C. and at pH 5 or 6. Enzymatic activity is defined as mM total reducing sugars following a 30-min reaction period. Each reaction was performed in triplicate, and standard deviations are indicated.

FIGS. 2A-B are photographs of Western-blot analysis of culture supernatants from transformed lactobacilli. A. Lanes 1 to 3: Cel6A expressed with the Lp1, Lp2 and No-Lp plasmids, respectively. B. Lanes 1 to 3: Xyn11A expressed with the Lp1, Lp2 and No-Lp plasmids, respectively. The calculated masses of secreted Cel6A and Xyn11A are 46.9 kDa and 33.2 kDa, respectively. The lane of the prestained molecular weight markers in Panel A was manually inserted as a reference onto the chemiluminescent image of the blot.

FIGS. 3A-D are bar graphs illustrating quantification of the secreted enzymes. A. Dot blot analysis of increasing concentrations of purified Cel6A in nM and 2 μL of concentrated culture supernatant fluids. The graph shows the mean intensity of each spot for the calibration curve in black and the white circles represent the intensity of the spot for Cel6A cultures (No-Lp, Lp1 and Lp2). B. Dot blot analysis of increasing concentrations of purified Xyn11A in nM and 2 μL of dialyzed culture supernatant fluids. The irrelevant spots between the samples of Lp2 and No-Lp were cropped in the panel. C. Enzymatic activity on PASC. Reactions were conducted with increasing concentrations of purified Cel6A and with 30 μL concentrated culture supernatant fluids. Enzymatic activity is defined as mM soluble reducing sugars released following an 18-h reaction period. Each reaction was performed in triplicate, and standard deviations are indicated. D. Enzymatic activity on xylan. Reactions were conducted with increasing concentrations of purified Xyn11A and with 30 μL dialyzed culture supernatant fluids. Enzymatic activity is defined as mM soluble reducing sugars following a 2-h reaction period. Each reaction was performed in triplicate, and standard deviations for xylan hydrolysis are indicated.

FIG. 4 is a bar graph illustrating activity of secreted enzymes on various substrates. Comparative enzymatic activity of supernatants derived from cultures producing either the cellulase (grey bars) or the xylanase (white bars). The substrates, PASC, xylan or pretreated wheat straw, were incubated with 30 μl of supernatant fluids (concentrated to approximately 16.5 nM of enzyme). The enzymatic activity of Cel6A is represented by grey bars and Xyn11A by white bars. Enzymatic activity is defined as mM soluble reducing sugars following a 2-h reaction period for xylan, 18-h incubation for PASC or 24-h incubation for wheat straw at pH 5 and 37° C. Each reaction was performed in triplicate, and standard deviations are indicated.

FIG. 5 is a bar graph illustrating enzymatic activity in supernatants of co-cultures producing the cellulase and the xylanase. The substrate was pretreated wheat straw and the measured activities are compared with the corresponding theoretical additive effect. Cells were inoculated using various ratios (Cel6A/Xyn11A): 1/500, 1/100, 1/50, 1/10, 1/1, 10/1, 50/1, 100/1 and 500/1 (where the 10/1 cell ratio corresponds to an approximate 1:1 molar ratio of the secreted enzymes, since cellulase production is approximately 10-fold lower; see text and FIG. 3). The concentration of pretreated wheat straw (dry matter) in the reactions was 3.5 g/l. Assuming that all detected reducing ends belong to dimers the highest detected product concentration (1:500 ratio) represents 27.6% polysaccharide conversion. Enzymatic activity is defined as mM soluble reducing sugars following a 24-h reaction period at 37° C. and pH 5. Each reaction was performed in triplicate, and standard deviations are indicated. The theoretical additive effect is defined as the sum of the activities of the individual Cel6A- and Xyn11A-secreting cultures (see Materials and Methods section for a detailed explanation), and synergism was calculated as the ratio between the measured activity and the theoretical activity assuming additivity.

FIG. 6 is a bar graph illustrating the ratios between Lp1-Cel6A and Lp2-Xyn11A in cocultures after the growth period, as determined by RT-PCR. Cultures were inoculated using various ratios (Cel6A/Xyn11A): 1/500, 1/100, 1/50, 1/10, 1/1, 10/1, 50/1, 100/1 and 500/1. Total copy numbers of each plasmid were determined for each coculture, and ratios were calculated.

### DESCRIPTION OF SPECIFIC EMBODIMENTS OF THE INVENTION

The present invention, in some embodiments thereof, relates to lactic acid bacteria genetically modified to express enzymes of the cellulotyic system and, more particularly, but not exclusively, to a cellulase and a xylanase.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In ensiled crops, lactic acid bacteria convert low molecular weight carbohydrates into lactic acid, which is the main preservative in ensilage. In some crops it is important to add lactic acid bacteria as an inoculums during ensiling in order to boost fermentation in the initial stages.

In crops such as grass or Lucerne, the low amount of fermentable carbohydrate limits lactic fermentation, which leads to unreliable preservation of the silage. This can be artificially improved by adding sources of fermentable carbohydrates, or enzymes which release the latter from, for example, plant fiber. Although cellulase is sold and used as a commercial silage additive, its application is expensive. Therefore the introduction of lignocellulose processing enzymes, such as cellulase and xylanase in lactic acid bacteria has been proposed.

The present inventors propose transformation with a synergistic combination of cellulase and xylanase genes which enable lactic acid bacteria to release higher amounts of fermentable carbohydrates from ensiled crops, thereby improving silage quality.

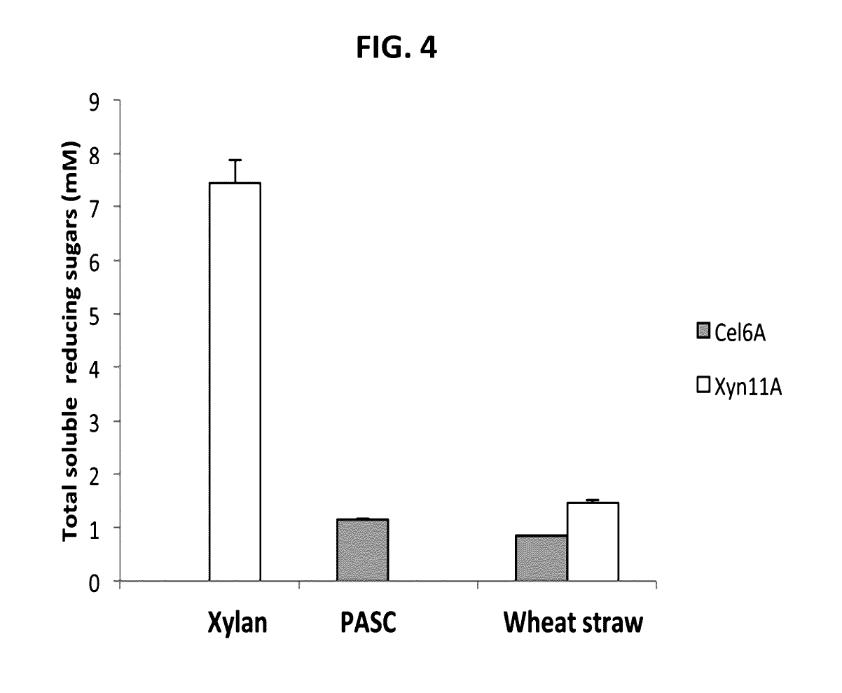

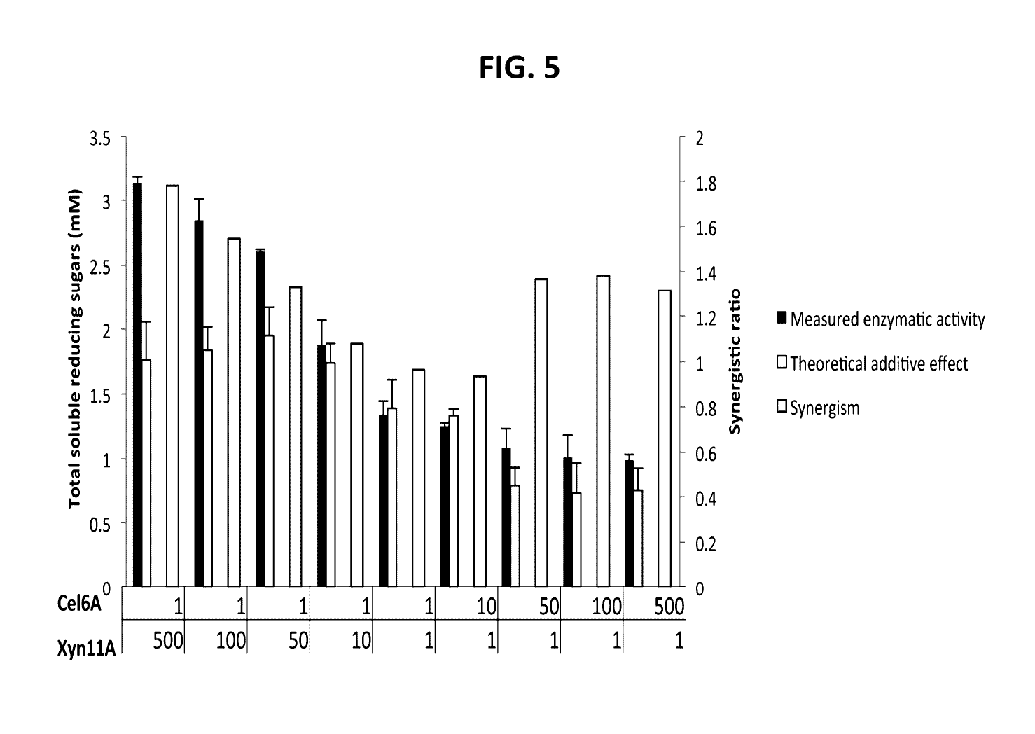

Whilst reducing the present invention to practice, the present inventors generated two individual populations of lactic acid bacteria, the first being genetically engineered to express cellulase and the second being genetically engineered to express xylanase. The enzymatic activity of each individual population was confirmed on cellulose and xylan respectively (FIG. 4). When mixed together to form a two-strain cell-based consortium secreting both cellulase and xylanase, they exhibited synergistic activity in the overall release of soluble sugar from wheat straw (FIG. 5). Synergistic activities (>1) were observed for molar ratios of 1/5 and greater, in favor of bacteria secreting either enzyme. The highest overall activities and the largest synergistic effect were observed in reactions with a strong dominance of the Xyn11A-secreting strain, reaching a synergy factor of 1.8, and yielded up to 27.6% of available sugars.

Thus, according to one aspect of the present invention there is provided a bacterial culture comprising a biomass composition and a population of lactic acid bacteria which comprises:

(i) a first population of lactic acid bacteria which has been genetically modified to express a secreted cellulase; and

(ii) a second population of lactic acid bacteria which has been genetically modified to express a secreted xylanase, wherein the ratio of the first population:second population is selected such that the specific activity of cellulase:xylanase in the culture is greater than 4:1 or less than 1:4.

As used herein, the phrase “lactic acid bacterium” refers to a group of gram-positive, microaerophilic or anaerobic bacteria having in common the ability to ferment sugars and citrate with the production of acids including lactic acid as the predominantly produced acid, acetic acid, formic acid and propionic acid.

Examples of lactic acid bacteria include, but are not limited to Lactobacillus plantarum, Lactococcus lactis, Leuconostoc mesenteroides, Streptococcus thermophilus, Pediococcus pentosaceus and Lactobacillus acidophilus.

The term “cellulase” refers to both endoglucanases and exoglucanases. Endoglucanases randomly cleave cellulose chains into smaller units. Exoglucanases include cellobiohydrolases, which liberate glucose dimers (cellobiose) from the ends of cellulose chains; glucanhydrolases, which liberate glucose monomers from the ends of cellulose chains; and, beta-glucosidases, which liberate D-glucose from cellobiose dimers and soluble cellodextrins.

The term “exoglucanase”, “exo-cellobiohydrolase” or “CBH” refers to a group of cellulase enzymes classified as E.C. 3.2.1.91. These enzymes hydrolyze cellobiose from the reducing or non-reducing end of cellulose. Exo-cellobiohydrolases include, but are not limited to, enzymes classified in the GH5, GH6, GH7, GH9, and GH48 GH families.

The term “endoglucanase” or “EG” refers to a group of cellulase enzymes classified as E.C. 3.2.1.4. These enzymes hydrolyze internal beta-1,4 glucosidic bonds of cellulose. Endoglucanases include, but are not limited to, enzymes classified in the GH5, GH6, GH7, GH8, GH9, GH12, GH44, GH45, GH48, GH51, GH61, and GH74 GH families.

The term “xylanase” refers to the class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. Xylanases include, but are not limited to, enzymes classified in the GHS, GH8, GH10, GH11, and GH43 GH families.

The skilled person will appreciate that enzymes having cellulase or xylanase activity isolated from a variety of sources may be used in the present invention.

Further, it will be appreciated that the sequences of the cellulases and xylanases which are expressed in the lactic acid bacteria of the present invention do not necessarily have to be 100% homologous to the sequences from their source organisms.

Thus, enzymes which are expressed in the lactic acid bacteria of the present invention may be homologs and other modifications including additions or deletions of specific amino acids to the sequence (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to the native amino acid sequences of the source organisms, as determined using BlastP software of the National Center of Biotechnology Information (NCBI) using default parameters). The homolog may also refer to an ortholog, a deletion, insertion, or substitution variant, including an amino acid substitution, thereof and biologically active polypeptide fragments thereof.

Some examples of suitable cellulase enzymes include, but are not limited to, those from the thermophilic bacteria Thermobifida fusca (DNA: SEQ ID NO:12, protein: SEQ ID NO:13), Acidothermus cellulolyticus (protein: SEQ ID NO:28), Thermobispora bispora (DNA: SEQ ID NO:29, protein: SEQ ID NO:30) and Themomonospora curvata (DNA: SEQ ID NO:31, protein: SEQ ID NO:32).

Some examples of suitable xylanase enzymes include, but are not limited to, those from the thermophilic bacteria Thermobifida fusca (DNA: SEQ ID NO:14, protein: SEQ ID NO:15), Clostridium clariflavum (DNA: SEQ ID NO:18, protein: SEQ ID NO:19), Clostridium thermocellum (DNA: SEQ ID NO:20, protein: SEQ ID NO:21) Thermobifida halotolerans (DNA: SEQ ID NO:22, protein: SEQ ID NO:23) Thermobispora bispora (DNA: SEQ ID NO:24, protein: SEQ ID NO:25), Thermopolyspora flexuosa (DNA: SEQ ID NO:26, protein: SEQ ID NO:27).

Because cellulase and xylanase enzymes are well known, and because of the prevalence of genomic sequencing, suitable cellulases and xylanases may be readily identified by one skilled in the art on the basis of sequence similarity using bioinformatics approaches.

It is well understood by one skilled in the art that many levels of sequence identity are useful in identifying polypeptides, from other species, wherein such polypeptides have the same or similar function or activity. Useful examples of percent identities include, but are not limited to: 24%, 30%, 35%, 40%, 45%, 50%, 55%, 60%, 65%, 70%, 75%, 80%, 85%, 90%, or 95%, or any integer percentage from 24% to 100% may be useful in describing the present invention, such as 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Bioinformatic approaches typically comprise the use of sequence analysis software which may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mish.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Typically BLAST (described above) searching of publicly available databases with known cellulase/xylanase amino acid sequences, such as those provided herein, is used to identify cellulase/xylanase, and their encoding sequences, that may be used in the present strains.

Thus, the present inventors also contemplate cellulases and xylanases from fiber-degrading bacteria that are the inhabitants of a ruminant's gut ecosystem. Such bacteria include Ruminococcus flavefaciens or Ruminococcus albus, the genome of strains of each of these species have already been sequenced and partially characterized [Rincon et al, 2010, PLoS ONE 5, e12476].

Alternative sources of appropriate enzymes from other mesophilic environmental (but non-ruminant) sources are also contemplated. These include enzymes from the following cellulosome-producing bacteria: Acetivibrio cellulolyticus, Bacteroides cellulosolvens, Clostridium papyrosolvens and Clostridium cellulolyticum.

According to one embodiment, the cells may have a xylose conversion yield of at least 1 to 29%.

Cells of the invention may have a specific xylose degradation rate of at least about 200, about 250, about 300, about 346, about 350, about 400, about 500, about 600, about 750, or about 1000 mg xylose/g cells/h.

According to one embodiment, the cells may have a cellulose conversion yield of at least 1 to 8%.

Cells of the invention may have a specific cellulose degradation rate of at least about 200, about 250, about 300, about 346, about 350, about 400, about 500, about 600, about 750, or about 1000 mg cellulose/g cells/h.

Expression of heterologous enzymes such as cellulase and xylanase is achieved by transforming suitable host cells with a polynucleotide sequence encoding the enzyme. Typically the coding sequence is part of a chimeric gene used for transformation, which includes a promoter operably linked to the coding sequence as well as a ribosome binding site and a termination control region. A chimeric gene is heterologous even if it includes the coding sequence for the enzyme from the host cell for transformation, if the coding sequence is combined with regulatory sequences that are not native to the natural gene encoding the enzyme.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a bacterial cell.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

Codon degeneracy refers to the nature in the genetic code permitting variation of the nucleotide sequence without effecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

Thus, codons may be optimized for expression based on codon usage in the selected host, as is known to one skilled in the art.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

Vectors useful for the transformation of a variety of host cells are common and described in the literature. Typically the vector contains a selectable marker and sequences allowing autonomous replication or chromosomal integration in the desired host. In addition, suitable vectors may comprise a promoter region which harbors transcriptional initiation controls and a transcriptional termination control region, between which a coding region DNA fragment may be inserted, to provide expression of the inserted coding region. Both control regions may be derived from genes homologous to the transformed host cell, although it is to be understood that such control regions may also be derived from genes that are not native to the specific species chosen as a production host.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Initiation control regions or promoters, which are useful to drive expression of a cellulase or xylanase coding region in lactic acid bacteria (LAB) are familiar to those skilled in the art. Some examples include the amy, apr, and npr promoters; nisA promoter (useful for expression Gram-positive bacteria (Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). In addition, the ldhL1 and fabZ1 promoters of L plantarum are useful for expression of chimeric genes in LAB. The fabZ1 promoter directs transcription of an operon with the first gene, fabZ1, encoding (3R)-hydroxymyristoyl-[acyl carrier protein] dehydratase.

Termination control regions may also be derived from various genes, typically from genes native to the preferred hosts. Optionally, a termination site may be unnecessary.

Vectors useful in LAB include vectors having two origins of replication and two selectable markers which allow for replication and selection in both Escherichia coli and LAB. An example is pFP996, which is useful in L. plantarum and other LAB. Many plasmids and vectors used in the transformation of Bacillus subtilis and Streptococcus may be used generally for LAB. Non-limiting examples of suitable vectors include pAM beta1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (e.g., van Kranenburg R, Golic N, Bongers R, Leer R J, de Vos W M, Siezen R J, Kleerebezem M. Appl. Environ. Microbiol. 2005 March; 71(3): 1223-1230).

According to a particular embodiment, the vector is based on the pSIP system which are further described in Sorvig et al., 2005, Microbiology 151:2439-2449; and Mathiesen G et al., Journal of applied microbiology 105:215-226.

Standard recombinant DNA and molecular cloning techniques used in the generation of vectors suitable for use in the present invention are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

Since the present invention contemplates expression of secreted cellulase and xylanase, typically the polynucleotides encoding the enzymes encode a pre-protein form of the enzymes.

The term “pre-protein” refers to a secreted protein with an amino-terminal signal peptide region attached. The signal peptide is cleaved from the pre-protein by a signal peptidase prior to secretion to result in the “mature” or “secreted” protein. The signal peptide may or may not be heterologous to the particular enzyme sequence.

Exemplary signal peptides include those that are derived from the L. plantarum WCFS1 proteins pLp_2145s (SEQ ID NO: 16) and pLp_3050s (SEQ ID NO: 17).

Vectors may be introduced into a host cell using methods known in the art, such as electroporation (Cruz-Rodz et al. Molecular Genetics and Genomics 224:1252-154 (1990), Bringel, et al. Appl. Microbiol. Biotechnol. 33: 664-670 (1990), Alegre et al., FEMS Microbiology letters 241:73-77 (2004)), and conjugation (Shrago et al., Appl. Environ. Microbiol. 52:574-576 (1986)). A chimeric cellulase or xylanase gene can also be integrated into the chromosome of LAB using integration vectors (Hols et al., Appl. Environ. Microbiol. 60:1401-1403 (1990), Jang et al., Micro. Lett. 24:191-195 (2003)).

As mentioned, the present invention contemplates the use of two populations of lactic acid bacteria, the first population being genetically modified to express a cellulase and the second population being genetically modified to express a xylanase. The two populations may comprise identical strains of lactic acid bacteria. Thus, for example the first population of bacteria may comprise L. plantarum genetically modified to express a cellulase and the second population of bacteria may comprise L. plantarum genetically modified to express a xylanase. Alternatively, the two populations may comprise non-identical strains of lactic acid bacteria. Thus, for example the first population of bacteria may comprise L. plantarum genetically modified to express a cellulase and the second population of bacteria may comprise L. Lactus genetically modified to express a xylanase (or vice versa).

It will be appreciated also, that the first and second bacterial cell population may or may not be a pure population (i.e. comprise a single strain of bacteria) but may be a mixed population of two or more strains of bacteria.

The two populations may be provided in a single article of manufacture, each population being individually packaged.

According to another aspect of the present invention there is provided a population of lactic acid bacteria which have been genetically modified to express both a secreted cellulase and a secreted xylanase.

Cultures of the two individual bacterial cell population (i.e. the first modified to express a cellulase and the second modified to express a xylanase) or the single population (i.e. one population modified to express both a cellulase and a xylanase) are contemplated by the present inventors.

The cultures comprise a biomass composition and optionally a fermentation media.

As used herein, the term “biomass composition” refers to biological material which comprise cellulose and xylan (or other polymers that may be degraded to produce cellulose or xylan).

According to one embodiment, the biomass composition comprises cellulose and hemicellulose.

Cellulose is the most abundant polymer of the plant cell wall, constituting 30-40% of its content. Second are the hemicelluloses constituting 20-25%. Cellulose polymers are composed of D-glucose subunits attached in linear fashion by β-(1-4) glycosidic bonds. The repeating dimers of glucose are named cellobiose and are considered as the basic cellulose subunits. Hemicellulose is composed of a versatile array of branched sugar polymers, among which xylan is the most abundant. Two units of D-xylose monomers attached by a β-(1-4) glycosidic bond constitute the basic subunit of xylan named xylobiose. In addition to these basic units, xlyan usually contains various sugar side chains attached to it. Together these two polymers make up most of the plant cell wall.

The biological material may be living or dead. The biomass composition may further include lignocelluloses, hemicellulose, lignin, mannan, and other materials commonly found in biomass. Non-limiting examples of sources of a biomass composition include grasses (e.g., switchgrass, Miscanthus), rice hulls, bagasse, cotton, jute, eucalyptus, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe). Sources of a biomass polymer may be an unrefined plant feedstock (e.g., ionic liquid-treated plant biomass) or a refined biomass polymer (e.g., beechwood xylan or phosphoric acid swollen cellulose). Additional sources of biomass composition include paper, paper products, paper waste, wood, particle board, sawdust, agricultural waste, sewage, silage, grasses, rice hulls, bagasse, jute, hemp, flax, bamboo, sisal, abaca, straw, corn cobs, corn stover, switchgrass, alfalfa, hay, coconut hair, cotton, seaweed, algae, and mixtures thereof.

In addition to the biomass material, the fermentation media may contain suitable minerals, salts, cofactors, buffers and other components, known to those skilled in the art, suitable for the growth of the cultures. Typically cells are grown at a temperature in the range of about 25° C. to about 40° C. in an appropriate medium. Suitable media for growing lactic acid bacteria are known in the art. Selection of a medium for growth of a particular bacterial strain will be known by one skilled in the art of microbiology or fermentation science. The use of agents known to modulate catabolite repression directly or indirectly, e.g., cyclic adenosine 2′:3′-monophosphate, may also be incorporated into the fermentation medium.

An exemplary medium which may be used for propagating the genetically modified bacteria may comprise at least 10, at least 20, at least 30, at least 40, at least 50 or all of the components listed in Table 1 herein below—see for example Wegkamp et al, Letters in Applied Microbiology, 50 (2010) 57-64. Table 1 also provides exemplary concentrations of the components.

TABLE 1

| Medium component | g/l |
|---|---|
| K₂HPO₄ | 21.68 |
| KH₂PO₄ | 12.93 |
| Glucose | 11 |
| Sodium acetate (*3H₂O) | 1.0 |
| | (1.65) |
| Ammonium citrate | 0.6 |
| Ascorbic acid | 0.5 |
| Alanine | 0.24 |
| Arginine | 0.125 |
| Aspartic acid | 0.42 |
| Cysteine | 0.13 |
| Glutamate | 0.5 |
| Glycine | 0.175 |
| Histidine | 0.15 |
| Isoleucine | 0.21 |
| Leucine | 0.475 |
| Lysine | 0.44 |
| Methionine | 0.125 |
| Phenylalanine | 0.275 |
| Proline | 0.675 |
| Serine | 0.34 |
| Threonine | 0.225 |
| Tryptophane | 0.05 |
| Tyrosine | 0.25 |
| Valine | 0.325 |
| 6,8-thiotic acid (α-lipoic acid) | 0.001 |
| Biotin | 0.0025 |
| Nicotinic acid | 0.001 |
| Panthothenic acid (Ca-pantothenate) | 0.001 |
| Para-aminobenzoic acid | 0.01 |
| Pyridoxamine | 0.005 |
| Pyridoxine | 0.002 |
| Riboflavin | 0.001 |
| Thiamine | 0.001 |
| Vitamin B12 | 0.001 |
| Adenine | 0.01 |
| Guanine | 0.01 |
| Inosine | 0.005 |
| Xanthine | 0.01 |
| Orotic acid | 0.005 |
| Thymidine | 0.005 |
| Uracil | 0.01 |
| MgCl₂ (*6H₂O) | 0.02 |
| | (0.426) |
| CaCl₂ (*2H₂O) | 0.05 |
| | (0.066) |
| MnCl₂ (*2H₂O) | 0.016 |
| | (0.02) |
| FeCl₃ (*6H₂O) | 0.003 |
| | (0.005) |
| FeCl₂ (*4H₂O) | 0.005 |
| | (0.0078) |
| ZnSO₄ | 0.005 |
| CoSO₄ (CoCl₂*6H₂O) | 0.0025 |
| | (0.003) |
| CuSO₄ | 0.0025 |
| (NH₄)6Mo₇O₂₄ (*4H₂O) | 0.0025 |
| | (0.0026) |

Suitable pH ranges for the fermentation are between pH 4.5 to pH 7.0, where pH 5.0 to pH 6.0 is preferred as the initial condition.

Fermentations may be performed under aerobic or anaerobic conditions, where anaerobic or microaerobic conditions are preferred.

The relative ratio of each of the populations in the culture is selected such that the specific activity of cellulase:xylanase in the culture is greater than 4:1 or less than 1:4.

According to a specific embodiment, the specific activity of cellulase:xylanase in the culture is greater than 4:1.

According to a specific embodiment, the specific activity of cellulase:xylanase in the culture is greater than 5:1.

According to a specific embodiment, the specific activity of cellulase:xylanase in the culture is greater than 6:1.

According to a specific embodiment, the specific activity of cellulase:xylanase in the culture is greater than 7:1.

According to a specific embodiment, the specific activity of cellulase:xylanase in the culture is greater than 8:1.

According to a specific embodiment, the specific activity of cellulase:xylanase in the culture is greater than 9:1.

According to a specific embodiment, the specific activity of cellulase:xylanase in the culture is greater than 10:1.

According to a specific embodiment, the specific activity of cellulase:xylanase in the culture is greater than 20:1.

According to a specific embodiment, the specific activity of cellulase:xylanase in the culture is greater than 30:1.

According to a specific embodiment, the specific activity of cellulase:xylanase in the culture is greater than 40:1.

According to a specific embodiment, the specific activity of cellulase:xylanase in the culture is greater than 50:1.

According to a specific embodiment, the specific activity of xylanase:cellulase in the culture is greater than 4:1.

According to a specific embodiment, the specific activity of xylanase:cellulase in the culture is greater than 5:1.

According to a specific embodiment, the specific activity of xylanase:cellulase in the culture is greater than 6:1.

According to a specific embodiment, the specific activity of xylanase:cellulase in the culture is greater than 7:1.

According to a specific embodiment, the specific activity of xylanase:cellulase in the culture is greater than 8:1.

According to a specific embodiment, the specific activity of xylanase:cellulase in the culture is greater than 9:1.

According to a specific embodiment, the specific activity of xylanase:cellulase in the culture is greater than 10:1.

According to a specific embodiment, the specific activity of xylanase:cellulase in the culture is greater than 20:1.

According to a specific embodiment, the specific activity of xylanase:cellulase in the culture is greater than 30:1.

According to a specific embodiment, the specific activity of xylanase:cellulase in the culture is greater than 40:1.

According to a specific embodiment, the specific activity of xylanase:cellulase in the culture is greater than 50:1.

The term “specific activity” as used herein refers to the amount of substrate consumed and/or product produced in a given time period and per defined amount of protein at a defined temperature.

The culture of the present invention may also comprise enzymes capable of degrading lignin. Such enzymes include phenol oxidases such as lignin peroxidases (LiP), manganese peroxidases (MnP) and laccases which may be comprised in white-rot fungi such as P. chrysosporium, Pleurotus ostreatus and Trametes versicolor. Laccase has broad substrate specificity and oxidises phenols and lignin substructures with the formation of oxygen radicals. Other enzymes that participate in the lignin degradation processes are H₂O₂-producing enzymes and oxido-reductases, which can be located either intra- or extracellularly. Bacterial and fungal feruloyl and p-coumaroyl esterases are relatively novel enzymes capable of releasing feruloyl and p-coumaroyl and play an important role in biodegradation of recalcitrant cell walls in grasses.

The cultures of the present invention may be used for processing lignocelluloses. Following metabolism of the lignocellulose, the released pentose sugars may be used for generating products such as biofuels (e.g. ethanol, butanol) or polylactic acid.

Since a dominant lactic acid fermentation is the key to making good silage, the cultures of the present invention may be used in silage products. Silage is the material produced by controlled fermentation of crop residues or forages with high moisture content. The purpose is to preserve forages by natural fermentation by achieving anaerobic conditions and discouraging clostridial growth.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Material and Methods

Cloning: Wild-type enzymes Cel6A and Xyn11A were cloned from Thermobifida fusca genomic DNA as described previously (21, 22). The enzyme constructs in pET28a were designed to contain a His-tag for subsequent purification.

For expression and secretion in L. plantarum, the glycoside hydrolases were cloned in the modular secretion plasmids pLp_2145sAmy and pLp_3050sAmy (15) by replacing the amylase gene in these plasmids by an appropriately amplified gene fragment, using either SalI or XhoI (SalI is compatible with XhoI) and HindIII restriction sites. For this purpose the Cel6A-encoding gene was amplified using the forward primer 5′-tcttCTCGAGatggcatcccccagacctct-3′ (SEQ ID NO: 1) and reverse primer 5′-aatAAGCTTtcagctggcggcgcaggtaag-3′(SEQ ID NO: 2) (XhoI and HindIII sites in capital letters). The Xyn11A-encoding gene amplified cloned using 5′-tcttGTCGACatggccgtgacctccaacgag-3′ (SEQ ID NO: 3) and 5′-aatAAGCTTctagttggcgctgcaggaca-3′ (SEQ ID NO: 4) primers (SalI and HindIII sites in capital letters). The pLp_2145s constructs are referred to as Lp1, whereas the pLP_3050s containing constructs are referred to as Lp2.

pLp_2145sAmy and pLp_3050sAmy are part of the pSIP400 series (13). As a control the two enzymes were also cloned into pSIP407 (referred to as No-Lp), which contains the same replicon and promoter as Lp1 and Lp2 but lacks a leader peptide (13). To make these constructs, the pepN gene present in pSIP407 was replaced by an NcoI-XbaI fragment containing the cel6A gene or a BspHI-XbaI fragment containing the xyn11A gene, which leads to the gene being translationally fused to the promoter (BspHI is compatible with NcoI). For this purpose the Cel6A-encoding gene was amplified using the forward primer 5′-atatatCCATGGatggcatcccccagacctcttcgc-3′(SEQ ID NO: 5) and reverse primer 5′-atatatTCTAGAtcactccaggctggcggcgcagg-3′ (SEQ ID NO: 6; NcoI and XbaI sites in capital letters). The Xyn11A-encoding gene amplified cloned using 5′-tcagtcTCATGAatggccgtgacctccaacgagaccgg-3′ (SEQ ID NO: 7) and 5′-agcgtaTCTAGActagttggcgctgcaggacacc-3′ primers (SEQ ID NO: 8; BspHI and XbaI sites in capital letters).

For generation of empty pLP_2145s and pLP_3050s, the Amy gene was excised using SalI and EcoRI restriction enzymes. The linearized plasmid was purified and blunted using Quick Blunting Kit (NEB, MA, USA). Blunt fragments were self-ligated to create the empty plasmids.

PCR reactions were performed using Phusion High Fidelity DNA polymerase F530-S (New England Biolabs, Inc), and DNA samples were purified using a HiYield™ Gel/PCR Fragments Extraction Kit (Real Biotech Corporation, RBC, Taiwan). Restrictions enzymes were purchased from New England Biolabs (Beverly, Mass.) and the T4 DNA ligase from Fermentas (Vilnius, Lithuania). L. plantarum plasmids were sub-cloned in E. coli TG1 competent cells (Lucigen Corporation, WI, USA). L. plantarum strain WCFS1 was transformed according to the protocol of Aukrust et al (23). Antibiotics used for positive clone selection and added in media were 10 μg/ml and 200 μg/ml erythromycin for L. plantarum and E. coli, respectively.

Protein expression in E. coli, and purification: The plasmids pCel6A and pXyn11A were expressed in E. coli BL21 (1DE3) pLysS cells and the His-tagged enzymes were purified on a Ni-NTA column (Qiagen), as reported earlier (24). Purity of the recombinant proteins was tested by SDS-PAGE on 10% acrylamide gels, and fractions containing the pure recombinant protein were pooled and concentrated using Amicon centrifugal filters (Millipore, France). Protein concentrations were determined by measuring absorbance at 280 nm, using theoretical extinction coefficients calculated with the Protparam tool. Proteins were stored in 50% (v/v) glycerol at −20° C.

Activity assay for the pure enzymes: The activity of purified recombinant Cel6A and Xyn11A were tested in reactions containing 0.5 μM of enzyme and 7.5 g/l phosphoric acid-swollen cellulose (PASC, prepared as described by Lamed et al (25)) or 2% oat spelt xylan (Sigma Chem. Co, St. Louis Mo.) in 50 mM citrate buffer pH 5 or 6. Samples were incubated 30 min at 37 or 50° C., cooled to 0° C. by placing on ice, and then centrifuged 5 min at 14000 rpm at 4° C. The amount of soluble reducing sugars in the supernatants was determined by the DNS method as described below.

Protein expression in L. plantarum: Freshly inoculated cultures of L. plantarum WCFS 1 harboring a pSIP-derived expression plasmid was grown at 37° C. in MRS broth (BD Difco™, Franklin Lakes, N.J., USA) containing 10 μg/ml erythromycin). Gene expression was induced at an OD₆₀₀ of 0.3 by adding the inducing peptide for sakacin P production (Casio Laboratory, Denmark) (26) to a final concentration of 25 ng/ml and incubated for another 3 h at 37° C. For co-culture experiments, strains producing either the cellulase or the xylanase, respectively, were mixed at equal ODs or at various ratios and then grown and induced in the same manner.

Western-blot: Proteins from the culture supernatants were separated on SDS-PAGE gels (10% acrylamide) and transferred to a nitrocellulose membrane using Trans-Blot® Cell Mini (Bio-Rad Laboratories Ltd, Israel). Non-specific protein interactions were blocked by incubating the membrane for 1 h with 5% BSA (prepared in in Tris Buffer Saline-Tween 20, TBS-T). The membrane was then rinsed twice (1 min) with TBS-T. Rabbit antibody against each enzyme (prepared by Sigma, Israel) was incubated with the appropriate membrane for 1 h in TBS-T, containing 1% BSA. The membrane was again rinsed twice (1 min) with TBS-T and then incubated for 1-h with secondary antibody, mouse anti-rabbit horseradish peroxidase (HRP), at a dilution of 1:10000. The membrane was rinsed as described above and then rinsed twice (30 min) with TBS+1% Triton X-100. Blots were developed by incubating the membrane lmin with equal amounts of solution A & B of ECL (Ornat, Israel). Chemiluminescence was quantified using a luminescent image analyser, ImageQuant LAS 4000 Mini (Danyel Biotech, Israel).

Dot-blot: A volume of 50 ml cultures at OD₆₀₀=1, expressing the Cel6A enzyme (Lp1, Lp2 or No-Lp) was concentrated 50 times using Amicon centrifugal filters (Millipore, France). For the Xyn11A enzyme, 1 ml of each culture at OD₆₀₀=1 (Lp1, Lp2 or No-Lp) was dialyzed in TBS to remove MRS media. Purified enzymes were blotted in concentrations ranging from 0.5-20 nM for the cellulase or 0.1-10 nM for the xylanase by applying 2 μl of an appropriate solution (in TBS) to a nitrocellulose membrane (Whatman). Concentrated and/or dialyzed culture supernatants were blotted by applying 2 μl of cultures. The above-described protocol for the Western blot was then followed.

Congo-red assay: The protocol of Anbar was followed with modifications (27). Oat spelt xylan (0.3%) was used instead of carboxymethyl cellulose (CMC) for xylanase activity detection. Transformed L. plantarum cells were spread onto MRS plates containing erythromycin (10 μg/mL) and incubated overnight at 37° C. The plates were overlaid with 20 ml soft agar containing 0.3% (w/v) CMC or oat spelt xylan (for cellulase or xylanase activity detection), 0.7% agar and 200 μl of 0.1 μg/ml pSIP induction peptide in citrate buffer (25 mM, pH 5.0). The plates were incubated for 2 h at 37° C. to induce enzyme expression and activity. The plates were then stained for 10 min with fresh Congo red solution (0.25%) and destained in 1 M NaCl. Formation of halos around the colonies indicated production of endoglucanase or endoxylanase activity.

Activity assay: PASC degradation was assayed by mixing pure recombinant Cel6A varying from 0 to 100 nM (final concentration) or a volume of 30 μl of concentrated supernatants of the cultures (as described above) with 150 μl of 7.5 g/l phosphoric acid swollen cellulose (PASC) in a final volume of 200 μl 50 mM acetate buffer pH 5.0. Samples were incubated at 37° C. for 18 h, and the reactions were terminated by immersing the sample tubes in ice water. The samples were then centrifuged 2 min at 14000 rpm to remove the substrate.

The xylanase assay mixture consisted of 100 μl buffer (50 mM citrate buffer pH 6.0) with purified Xyn11A enzyme (0-5 nM) or a volume of 30 μl of dialyzed supernatants of the cultures in 50 mM of the same buffer. The reaction was commenced by adding 100 μl of 2% oat spelt xylan, and continued for 2 hours at 37° C. The reaction was stopped by transferring the tubes to an ice-water bath followed by centrifugation for 2 min at 14000 rpm.

Wheat straw (0.2-0.8 mm) provided by Valagro (Poitiers, France) was washed as described previously (28, 29). The material was then subjected to sodium hypochlorite (12%) pretreatment at room temperature for 1 h (30). The degradation assay was conducted in 200 μl 50 mM acetate/citrate buffer pH 5-6.0 containing 3.5 g/l of pretreated wheat straw and 30 μl of concentrated or dialyzed culture supernatants. In the case of supernatants from co-cultures (50 ml at OD₆₀₀=1) of strains secreting the Cel6A and Xyn11A enzymes, were concentrated 50 times using Amicon centrifugal filters (Millipore, France). Reactions were incubated for 24 h at 37° C.

All assays were performed in triplicate. Enzymatic activity was determined quantitatively by measuring the soluble reducing sugars released from the polysaccharide substrates by the dinitrosalicyclic acid (DNS) method (31, 32). DNS solution (150 μl) was added to 100 μl of sample, and after boiling the reaction mixture for 10 min, absorbance at 540 nm was measured. Sugar concentrations were determined using a glucose standard curve.

Evaluation of synergism: For determination of theoretical enzymatic activity in co-cultures (additive effect), enzymatic activities were calculated from two different assays. In each assay, a coculture of one of the enzyme-secreting strains together with the respective empty plasmid-bearing control strain was grown (and induced as described above), and its supernatant was analyzed for enzymatic activity. The theoretical additive activity was calculated by computing the sum of activities for each of the individually measured enzymes. For example, for the 1/500 ratio, one volume of the Cel6A-secreting strain (Lp1) and 500 volumes of the empty pLp_3050s plasmid-bearing strain (as a replacement for the Xyn11A-secreting strain (Lp2)) were cocultured. In parallel, one volume of the empty pLp_2145s plasmid-bearing strain (as a replacement for the Cel6A-secreting strain (Lp1)) and 500 volumes of the Xyn11A-secreting strain (Lp2) were cocultured. The enzymatic activities on wheat straw substrate of 30 μl of concentrated supernatants (as described above for the coculture experiments) from each of the cocultures were determined individually, added together and defined as the theoretical additive effect. These values were then compared with those of the corresponding combined cocultures of the cellulase- and xylanase-secreting strains.

Plasmid extraction: Cocultures of cellulase- and xylanase-secreting strains were grown as described above. At OD₆₀₀=1, cells were pelleted from 5 ml of culture by centrifugation at 5000 g for 10 min at 4° C. and resuspended in 200 μl of PD1 buffer of a High-Speed Plasmid Mini Kit (Geneaid, New Taipei City, TW). Lysozyme was added to the suspensions to a final concentration of 3 mg/ml. Suspensions were incubated at 37° C. for 15 min and then subjected to five freeze-thaw cycles as follows: the samples were submerged in liquid nitrogen for 3 min, transferred to 70° C. water bath for an additional 3 min and then mixed gently but thoroughly. Following this step, the protocol was carried out according to the manufacturer's instructions.

Real-time PCR: Quantitative real-time PCR analysis was performed to verify the ratios between the cellulase- and xylanase-secreting strains in the bacterial consortium. A specific fragment of each plasmid (140 and 124 bp for pLP_2145s and pLP_3050s respectively) was amplified using the forward primer 5′-ATTTAGCTGGCTGGCGTAAAGTATG-3′ (SEQ ID NO: 9) for both plasmids, and the reverse primers 5′-TCATTTCAGGATTGATCATTGTTGC-3′ (SEQ ID NO: 10) for pLP_2145s (Lp_1) and 5′-GACGACCCCGAAGACACAACTAG-3′ (SEQ ID NO: 11) for pLP_3050s (Lp2). Individual standard curves suitable for the quantification of each plasmid were generated by amplifying serial 10-fold dilutions of quantified gel-extracted PCR products obtained by the amplification of each fragment. The standard curves were obtained using four dilution points and were calculated using Rotorgene 6000 series software (Qiagen, Hilden, Germany). Subsequent quantifications were calculated with the same program using the standard curves generated. As positive control, one purified product with known concentration that was used for the standard curve was added to each quantification reaction. This also served to assess the reproducibility of the reactions and to fit the results to the standard curve. Two negative controls were performed; the first contained the purified product of one of the plasmids and the primers of the other. This was done in order to eliminate the possibility of primers cross-reactivity. The second control did not contain any DNA template. All obtained standard curves met the required standards of efficiency (R²>0.99, 90%<E<115%). The number of copies of each plasmid in the cultures was assessed and the ratio between the plasmids was determined. Real-time PCR was performed in a 10 μl reaction mixture containing 5 μl Absolute Blue SYBR Green Master Mix (Thermo Scientific, MA, USA), 0.5 μl of each primer (10 μM working concentration), 2 μl nuclease-free water and 2 μl of 10 ng/μl DNA template. Amplification involved one hold cycle at 95° C. for 15 min for initial denaturation and activation of the hot-start polymerase system, and then 30 cycles at 95° C. for 10 s followed by annealing for 20 s at 53.3° C. and extension at 72° C. for 20 s. To determine the specificity of amplification, a melting curve of PCR products was monitored by slow heating with fluorescence collection at 1° C. increments from 45 to 99° C.

Results

Choice of lignocellulolytic enzymes. The selected enzymes for L. plantarum transformation originate from the very well-characterized cellulolytic bacterium Thermobifida fusca. This bacterium produces a set of only six cellulases and four xylanases. These moderately thermophilic enzymes are known to have a broad temperature-activity and pH-activity (37), which might be compatible with the conditions expected during a Lactobacillus fermentation.

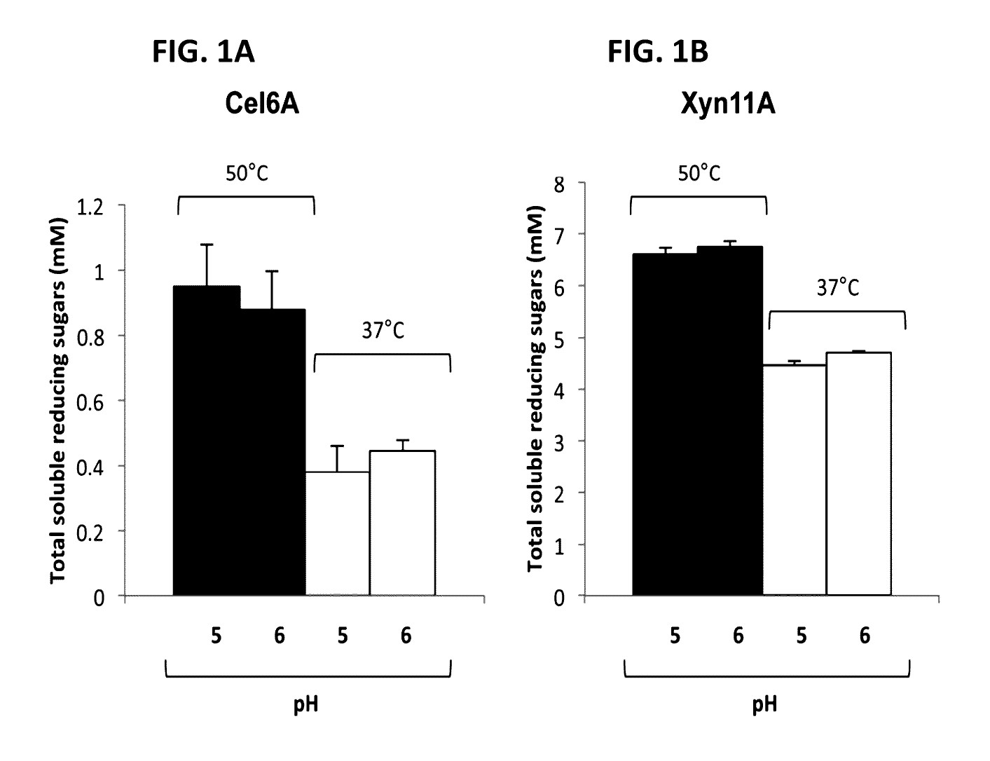

For initial studies, we focused on the T. fusca endoglucanase Cel6A, which is highly induced by cellobiose (38), and endoxylanase Xyn11A, which is the most abundant xylanase produced during growth on xylan (39). In addition to their catalytic modules, Cel6A has a C-terminal family 2 CBM which binds selectively to cellulose, and Xyn11A contains a C-terminal family 2 CBM that binds both cellulose and xylan. The molecular masses of the enzymes are 46,980 Da and 33,168 Da for Cel6A and Xyn11A, respectively. The selection of Cel6A and Xyn11A was also based on their simple modular architecture and their considerable residual activity under acidic conditions (activity at pH 5.0 is >90% of that at pH 6) and at 37° C. (˜40% and ˜70% of the activity at 50° C., for Cel6A and Xyn11A, respectively) (FIG. 1) consistent with normal growth of L. plantarum.

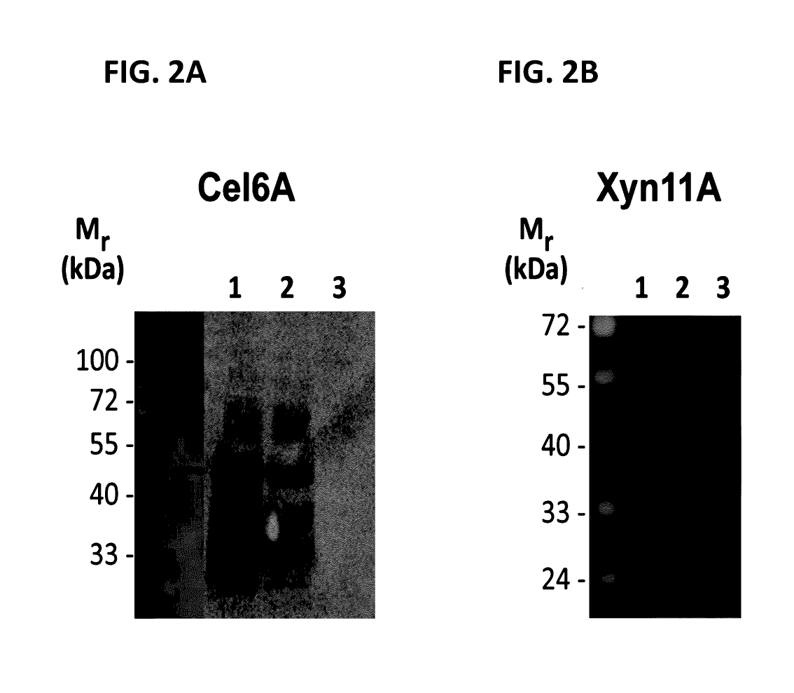

Enzyme secretion by L. plantarum. The presence of secreted enzymes in the culture medium was observed by Western Blotting using specific antibodies against each enzyme (FIGS. 2A-B). The enzymes were visible in the extracellular fraction of the strains carrying the Lp1 and Lp2 secretion plasmids, and the observed bands corresponded well to their theoretical masses. Degradation products, i.e. smaller bands, were also observed. No extracellular enzymes were detected in the supernatants of strains carrying the expression plasmid lacking the secretion peptide (FIG. 2, lane 3).

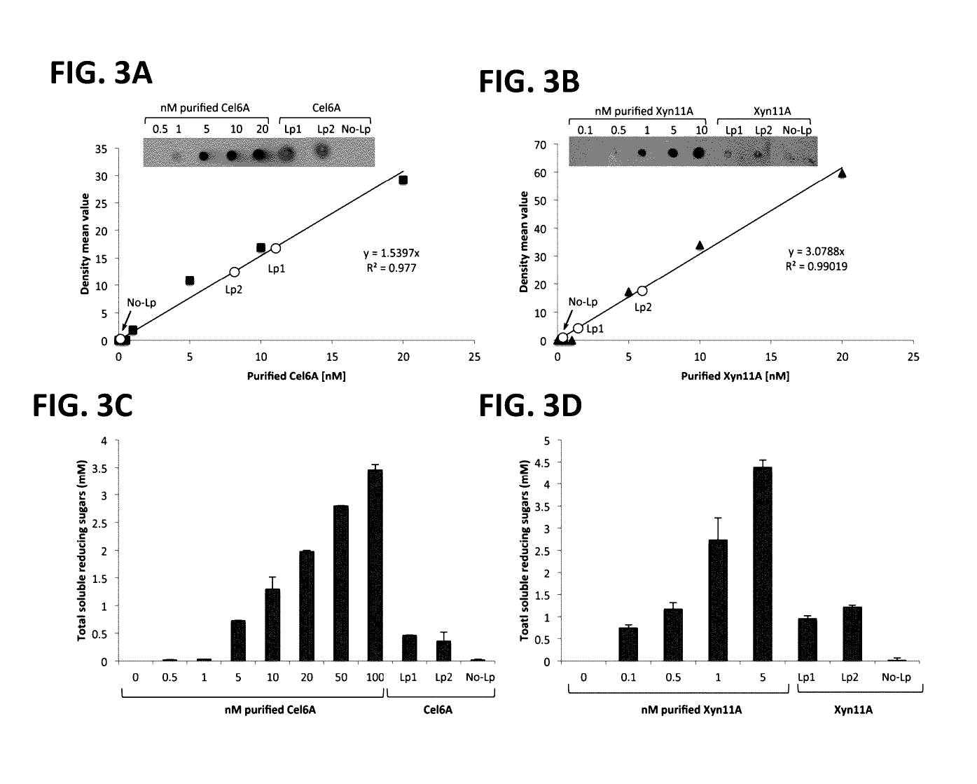

Extracellular cellulase and xylanase activities in transformed colonies were detected by the Congo-Red method (data not shown) and by activity assays of culture supernatants (FIGS. 3A-D; see below). Control cultures with intracellular expression of the respective enzymes did not exhibit any activity using the Congo-Red assay and their supernatants did not show hydrolytic activity on xylan or PASC.

The concentrations of the secreted enzymes in the different cultures were calculated by comparing the extracellular fraction to serial dilutions of purified enzymes, either by dot blot analysis or by measuring reducing sugar formation on PASC or xylan substrates. The cellulase concentrations at OD₆₀₀=1 were estimated at 0.33 nM and 0.27 nM for the Lp1 and Lp2 secretion plasmids, respectively. For the xylanase these values were estimated 2.7 nM and 3.3 nM, respectively (FIGS. 3C, D). The concentrations, calculated either by the dot-blot quantification or enzymatic activity method, were similar for both enzymes, suggesting that the major portion of the secreted enzymes is functional and that the expression and secretion processes do not substantially affect their activity. The culture supernatants retained full cellulase/xylanase activity after storage for several days at 4° C. without added protease inhibitors.

The fact that culture supernatants from strains with intracellular expression did not exhibit enzymatic activity (FIGS. 3C and D), indicates that the detected activities for the Lp1 and Lp2 constructs reflect properly secreted enzymes and do not originate from cell lysis.

Wheat straw degradation: Prior to enzymatic degradation, wheat straw was subjected to chemical pretreatment with sodium hypochlorite that served to reduce the lignin content while preserving the cellulose/hemicellulose fractions in order to promote enzymatic degradation. The chemical composition of the pretreated wheat straw was 63% cellulose, 31% hemicellulose and 3% lignin (30). Both the secreted cellulase and the secreted xylanase exhibited enzymatic activity on the pretreated wheat straw (FIG. 4).

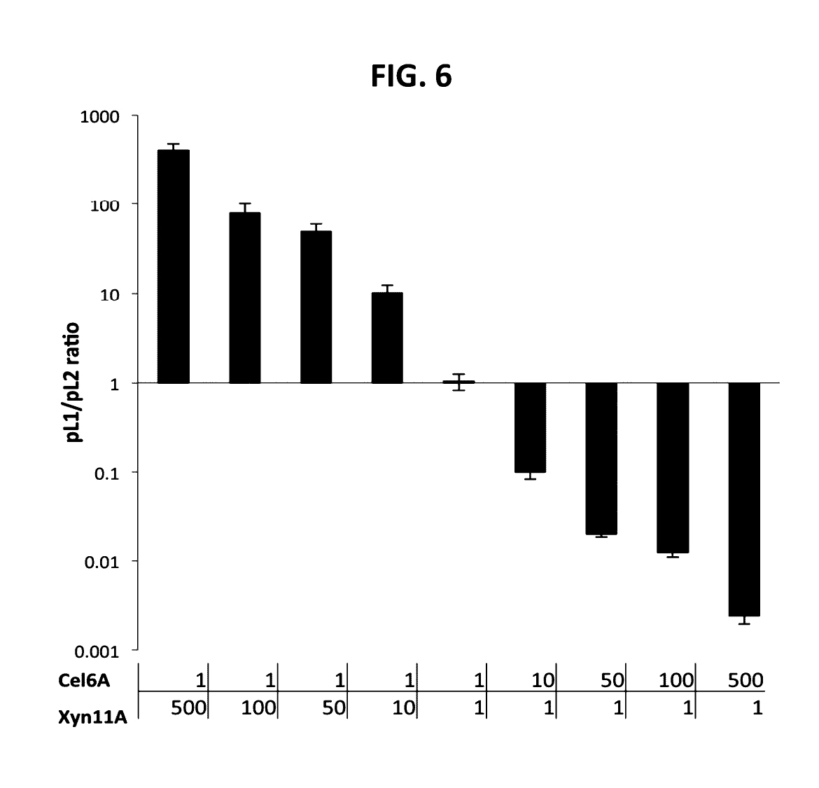

Supernatants of cocultures of a Cel6A-secreting strain (Lp1) and a Xyn_11A-secreting strain (Lp2) exhibited activity when incubated on wheat straw (FIG. 5). Synergistic activities (>1) were observed for ratios of 1/50 and greater, in favor of bacteria secreting either enzyme. The highest overall activities and the largest synergistic effect were observed in reactions with a strong dominance of the Xyn11A-secreting strain and yielded up to 27.6% of available sugars (FIG. 5), suggesting that xylan degradation by Xyn11A is a faster process than cellulose degradation by Cel6A. This observation further suggests that xylan degradation is more beneficial for cellulose accessibility than cellulose degradation is for xylan accessibility. RT-PCR of the different plasmids at the end of the growth period revealed that the ratios of the bacterial strains remained similar to the inoculation ratios (FIG. 6), thus indicating that expression and secretion of the two proteins did not have a differential effect on the growth rates of the bacteria.

Discussion

In this Example, the successful production and secretion of a cellulase and a xylanase by Lactobacillus plantarum is disclosed. Despite using identical cloning strategies, the enzymes were produced at different levels. An optimized cell consortium comprising two of the resulting strains was established using the efficiency of wheat straw degradation as the output parameter. These results provide a proof of principle for the engineering of lactobacilli for advanced biomass conversions. The T. fusca enzymes exhibit temperature optima ranging from 50-60° C. , but were nevertheless selected to their considerable residual activities at 37° C. and pH 5 (FIG. 1), i.e conditions that are common in L. plantarum cultures.

As a first step towards more complex biotransformations, the present inventors studied co-cultures of recombinant bacteria secreting the two enzymes. This approach was possible because the expression of the heterologous enzymes did not affect the bacterial growth, meaning that strain ratios remained rather stable during the growth period.

An advantage of using cocultures is that a cell consortium can easily be optimized by adjusting the ratio of each cell type during inoculation. In a recent publication, a mixture of S. cerevisiae cells with an optimized endoglucanase:exoglucanase:β-glucosidase ratio produced 1.3 fold more ethanol than cells composed of an equal amount of each cell type, suggesting the usefulness of a consortium of bacteria for lignocellulose bioprocessing (44). Such an approach can also be used to balance production levels, which may differ, as observed for Cel6A and Xyn11A in the present study.

The transformed L. plantarum cells were able to degrade either xylan or cellulose and wheat straw. Interestingly, co-culturing revealed clear synergistic effects with the synergy factor reaching 1.8 for combinations with a large excess of the xylanase. These results suggest that the action of the xylanase in deconstructing the substrate renders the cellulose accessible to the cellulase, as described in previous studies (45-47).

Several studies on other bacteria illustrate that L. plantarum producing these lignocellulolytic enzymes could have attractive applications. For example, integration of a cellulase from Bacillus sp. ATCC 21833 into the genome of L. plantarum led to increased efficiency in alfalfa silage fermentation (48). A similar results was reported for L. lactis strains transformed with a Neocallimastix sp. cellulase (49). The expression of genes coding for fibrolytic enzymes in lactobacilli is also of interest for the development of intestinal probiotic strains (50-52). Recently, co-expression of a β-glucanase and a xylanase in L. reuteri has been reported (52), and the transformed strain exhibited enzymatic activity on soluble β-glucan and xylan.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Bayer E A, Lamed R, White B A, Flint H J. 2008. From cellulosomes to cellulosomics. Chem Rec 8:364-377.
2. Teusink B, Wiersma A, Molenaar D, Francke C, de Vos W M, Siezen RJ, Smid E J. 2006. Analysis of growth of Lactobacillus plantarum WCFS 1 on a complex medium using a genome-scale metabolic model. The Journal of biological chemistry 281:40041-40048.
3. Klocke M, Mundt K, Idler C, McEniry J, O'Kiely P, Barth S. 2006. Monitoring Lactobacillus plantarum in grass silages with the aid of 16S rDNA-based quantitative real-time PCR assays. Systematic and applied microbiology 29:49-58.
4. Roach D R, Khatibi P A, Bischoff K M, Hughes S R, Donovan D M. 2013. Bacteriophage-encoded lytic enzymes control growth of contaminating Lactobacillus found in fuel ethanol fermentations. Biotechnology for biofuels 6:20.
5. Limayem A, Hanning I B, Muthaiyan A, Illeghems K, Kim J W, Crandall P G, O'Bryan C A, Ricke S C. 2011. Alternative antimicrobial compounds to control potential Lactobacillus contamination in bioethanol fermentations. Journal of environmental science and health. Part. B, Pesticides, food contaminants, and agricultural wastes 46:709-714.
6. Alegria E G, Lopez I, Ruiz J I, Saenz J, Fernandez E, Zarazaga M, Dizy M, Torres C, Ruiz□Larrea F. 2004. High tolerance of wild Lactobacillus plantarum and Oenococcus oeni strains to lyophilisation and stress environmental conditions of acid pH and ethanol. FEMS Microbiol Lett 230:53-61.
7. Nichols N N, Dien B S, Bothast R J. 2003. Engineering lactic acid bacteria with pyruvate decarboxylase and alcohol dehydrogenase genes for ethanol production from Zymomonas mobilis. Journal of industrial microbiology & biotechnology 30:315-321.
8. Domagk G F, Horecker B L. 1958. Pentose fermentation by Lactobacillus plantarum. V. Fermentation of 2-deoxy-D-ribose. The Journal of biological chemistry 233:283-286.
9. Kleerebezem M, Boekhorst J, van Kranenburg R, Molenaar D, Kuipers O P, Leer R, Tarchini R, Peters S A, Sandbrink H M, Fiers M W, Stiekema W, Lankhorst R M, Bron P A, Hoffer S M, Groot M N, Kerkhoven R, de Vries M, Ursing B, de Vos W M, Siezen R J. 2003. Complete genome sequence of Lactobacillus plantarum WCFS 1. Proceedings of the National Academy of Sciences of the United States of America 100:1990-1995.
10. Ganzle M G, Vermeulen N, Vogel R F. 2007. Carbohydrate, peptide and lipid metabolism of lactic acid bacteria in sourdough. Food microbiology 24:128-138.
11. Okano K, Yoshida S, Yamada R, Tanaka T, Ogino C, Fukuda H, Kondo A. 2009. Improved production of homo-D-lactic acid via xylose fermentation by introduction of xylose assimilation genes and redirection of the phosphoketolase pathway to the pentose phosphate pathway in L-Lactate dehydrogenase gene-deficient Lactobacillus plantarum. Appl Environ Microbiol 75:7858-7861.
12. Cantarel B L, Coutinho P M, Rancurel C, Bernard T, Lombard V, Henrissat B. 2009. The Carbohydrate-Active EnZymes database (CAZy): an expert resource for Glycogenomics. Nucleic Acids Res 37:D233-238.
13. Sorvig E, Mathiesen G, Naterstad K, Eijsink V G H, Axelsson L. 2005. High-level, inducible gene expression in Lactobacillus sakei and Lactobacillus plantarum using versatile expression vectors. Microbiology 151:2439-2449.
14. Sorvig E, Gronqvist S, Naterstad K, Mathiesen G, Eijsink V G H, Axelsson L. 2003. Construction of vectors for inducible gene expression in Lactobacillus sakei and L. plantarum. FEMS Microbiol Lett 229:119-126.
15. Mathiesen G, Sveen A, Brurberg M B, Fredriksen L, Axelsson L, Eijsink V G H. 2009. Genome-wide analysis of signal peptide functionality in Lactobacillus plantarum WCFS1. BMC genomics 10:425.
16. Mierau I, Kleerebezem M. 2005. 10 years of the nisin-controlled gene expression system (NICE) in Lactococcus lactis. Applied microbiology and biotechnology 68:705-717.
17. Kleerebezem M, Beerthuyzen M M, Vaughan E E, de Vos W M, Kuipers O P. 1997. Controlled gene expression systems for lactic acid bacteria: transferable nisin-inducible expression cassettes for Lactococcus, Leuconostoc, and Lactobacillus spp. Appl Environ Microbiol 63:4581-4584.
18. Pavan S, Hols P, Delcour J, Geoffroy M C, Grangette C, Kleerebezem M, Mercenier A. 2000. Adaptation of the nisin-controlled expression system in Lactobacillus plantarum: a tool to study in vivo biological effects. Appl Environ Microbiol 66:4427-4432.
19. Bohmer N, Lutz-Wahl S, Fischer L. 2012. Recombinant production of hyperthermostable CelB from Pyrococcus furiosus in Lactobacillus sp. Applied microbiology and biotechnology 96:903-912.
20. Mathiesen G, Sveen A, Piard J C, Axelsson L, Eijsink V G H. 2008. Heterologous protein secretion by Lactobacillus plantarum using homologous signal peptides. Journal of applied microbiology 105:215-226.
21. Morals S, Barak Y, Caspi J, Hadar Y, Lamed R, Shoham Y, Wilson D B, Bayer E A. 2010. Contribution of a xylan-binding module to the degradation of a complex cellulosic substrate by designer cellulosomes. Applied and Environmental Microbiology 76:3787-3796.
22. Ghangas G S, Wilson D B. 1988. Cloning of the Thermomonospora fusca Endoglucanase E2 Gene in Streptomyces lividans: Affinity Purification and Functional Domains of the Cloned Gene Product. Appl Environ Microbiol 54:2521-2526.
23. Aukrust T, Blom H. 1992. Transformation of Lactobacillus strains used in meat and vegetable fermentations. Food Res. Int. 25:253-261.
24. Caspi J, Irwin D, Lamed R, Shoham Y, Fierobe H-P, Wilson D B, Bayer E A. 2006. Thermobifida fusca family-6 cellulases as potential designer cellulosome components. Biocatalysis and Biotransformation 24:3-12.
25. Lamed R, Kenig R, Setter E, Bayer E A. 1985. Major characteristics of the cellulolytic system of Clostridium thermocellum coincide with those of the purified cellulosome. Enzyme Microb. Technol. 7:37-41.
26. Eijsink V G H, Brurberg M B, Middelhoven P H, Nes I F. 1996. Induction of bacteriocin production in Lactobacillus sakei by a secreted peptide. Journal of bacteriology 178:2232-2237.
27. Anbar M, Lamed R, Bayer E A. 2010. Thermostability enhancement of Clostridium thermocellum cellulosomal endoglucanase Cel8A by a single glycine substitution. ChemCatChem 2:997-1003.
28. Fierobe H-P, Mingardon F, Mechaly A, Belaich A, Rincon M T, Lamed R, Tardif C, Belaich J-P, Bayer E A. 2005. Action of designer cellulosomes on homogeneous versus complex substrates: Controlled incorporation of three distinct enzymes into a defined tri-functional scaffoldin. Journal of Biological Chemistry 280:16325-16334.
29. Tabka M G, Herpoel-Gimbert I, Monod F, Asther M, Sigoillot J C. 2006. Enzymatic saccharification of wheat straw for bioethanol production by a combined cellulase xylanase and feruloyl esterase treatment. Enzyme and Microbial Technology 39:897-902.
30. Morais S, Morag E, Barak Y, Goldman D, Hadar Y, Lamed R, Shoham Y, Wilson D B, Bayer E A. 2012. Deconstruction of lignocellulose into soluble sugars by native and designer cellulosomes. mBio 3.
31. Miller G L. 1959. Use of dinitrosalicylic acid reagent for determination of reducing sugar. Analytical Biochemistry 31:426-428.
32. Ghose T K. 1987. Measurments of cellulase activity. Pure. Appl. Chem 59:257-268.
33. Morals S, Barak Y, Caspi J, Hadar Y, Lamed R, Shoham Y, Wilson D B, Bayer E A. 2010. Cellulase-xylanase synergy in designer cellulosomes for enhanced degradation of a complex cellulosic substrate. mBio 1:e00285-00210.
34. Morais S, Morag E, Barak Y, Goldman D, Hadar Y, Lamed R, Shoham Y, Wilson D B, Bayer E A. 2013. Deconstruction of lignocellulose into soluble sugars by native and designer cellulosomes. mBio 3:e00508-00512.
35. Caspi J, Barak Y, Haimovitz R, Irwin D, Lamed R, Wilson D B, Bayer E A. 2009. Effect of linker length and dockerin position on conversion of a Thermobifida fusca endoglucanase to the cellulosomal mode. Applied and Environmental Microbiology 75:7335-7342.
36. Caspi J, Irwin D, Lamed R, Fierobe H-P, Wilson D B, Bayer E A. 2008. Conversion of noncellulosomal Thermobifida fusca free exoglucanases into cellulosomal components: Comparative impact on cellulose-degrading activity. Journal of Biotechnology 135:351-357.
37. Wilson D B. 2004. Studies of Thermobifida fusca plant cell wall degrading enzymes. Chem Rec 4:72-82.
38. Chen S, Wilson D B. 2007. Proteomic and transcriptomic analysis of extracellular proteins and mRNA levels in Thermobifida fusca grown on cellobiose and glucose. Journal of bacteriology 189:6260-6265.
39. Kim J H, Irwin D, Wilson D B. 2004. Purification and characterization of Thermobifida fusca xylanase 10B. Canadian Journal of Microbiology 50:835-843.
40. Nguyen T T, Nguyen T H, Maischberger T, Schmelzer P, Mathiesen G, Eijsink V G, Haltrich D, Peterbauer C K. 2011. Quantitative transcript analysis of the inducible expression system pSIP: comparison of the overexpression of Lactobacillus spp. beta-galactosidases in Lactobacillus plantarum. Microbial cell factories 10:46.
41. Irwin D, Jung E D, Wilson D B. 1994. Characterization and sequence of a Thermomonospora fusca xylanase. Applied and Environmental Microbiology 60:763-770.
42. McDonald L C, Fleming H P, Hassan H M. 1990. Acid Tolerance of Leuconostoc mesenteroides and Lactobacillus plantarum. Appl Environ Microbiol 56:2120-2124.
43. Mathiesen G, Huehne K, Kroeckel L, Axelsson L, Eijsink V G H. 2005. Characterization of a new bacteriocin operon in sakacin P-producing Lactobacillus sakei, showing strong translational coupling between the bacteriocin and immunity genes. Appl Environ Microbiol 71:3565-3574.
44. Baek S H, Kim S, Lee K, Lee J K, Hahn J S. 2012. Cellulosic ethanol production by combination of cellulase-displaying yeast cells. Enzyme Microb Technol 51:366-372.
45. Hu J, Arantes V, Saddler J N. 2011. The enhancement of enzymatic hydrolysis of lignocellulosic substrates by the addition of accessory enzymes such as xylanase: is it an additive or synergistic effect? Biotechnology for biofuels 4:36.
46. Qing Q, Wyman C E. 2011. Supplementation with xylanase and beta-xylosidase to reduce xylo-oligomer and xylan inhibition of enzymatic hydrolysis of cellulose and pretreated corn stover. Biotechnology for biofuels 4:18.
47. Zhang J, Tuomainen P, Siika-Aho M, Viikari L. 2011. Comparison of the synergistic action of two thermostable xylanases from GH families 10 and 11 with thermostable cellulases in lignocellulose hydrolysis. Bioresour Technol 102:9090-9095.
48. Rossi F, Rudella A, Marzotto M, Dellaglio F. 2001. Vector-free cloning of a bacterial endo-1,4-beta-glucanase in Lactobacillus plantarum and its effect on the acidifying activity in silage: use of recombinant cellulolytic Lactobacillus plantarum as silage inoculant. Antonie van Leeuwenhoek 80:139-147.
49. Ozkose E, Akyol I, Kar B, Comlekcioglu U, Ekinci M S. 2009. Expression of fungal cellulase gene in Lactococcus lactis to construct novel recombinant silage inoculants. Folia microbiologica 54:335-342.
50. Cho J S, Choi Y J, Chung D K. 2000. Expression of Clostridium thermocellum endoglucanase gene in Lactobacillus gasseri and Lactobacillus johnsonii and characterization of the genetically modified probiotic lactobacilli. Current microbiology 40:257-263.
51. Liu J R, Yu B, Liu F H, Cheng K J, Zhao X. 2005. Expression of rumen microbial fibrolytic enzyme genes in probiotic Lactobacillus reuteri. Appl Environ Microbiol 71:6769-6775.
52. Liu J R, Yu B, Zhao X, Cheng K J. 2007. Coexpression of rumen microbial beta-glucanase and xylanase genes in Lactobacillus reuteri. Applied microbiology and biotechnology 77:117-124.
53. Wood B E, Beall D S, Ingram L O. 1997. Production of recombinant bacterial endoglucanase as a co-product with ethanol during fermentation using derivatives of Escherichia coli KO11. Biotechnology and bioengineering 55:547-555.
54. Balusu R, Paduru R M, Seenayya G, Reddy G. 2004. Production of ethanol from cellulosic biomass by Clostridium thermocellum SS19 in submerged fermentation: screening of nutrients using Plackett-Burman design. Applied biochemistry and biotechnology 117:133-141.
55. Tsai S L, Oh J, Singh S, Chen R, Chen W. 2009. Functional assembly of minicellulosomes on the Saccharomyces cerevisiae cell surface for cellulose hydrolysis and ethanol production. Appl Environ Microbiol 75:6087-6093.

1. A bacterial culture comprising a biomass composition and a population of lactic acid bacteria which comprises:
(i) a first population of lactic acid bacteria which has been genetically modified to express a secreted cellulase; and
(ii) a second population of lactic acid bacteria which has been genetically modified to express a secreted xylanase, wherein the ratio of the first population: second population is selected such that the specific activity of cellulase:xylanase in the culture is greater than 4:1 or less than 1:4.

2. A population of lactic acid bacteria which have been genetically modified to express a secreted cellulase and a secreted xylanase.

3. A culture comprising the population of lactic acid bacteria of claim 2 and a biomass composition.

4. The culture of claim 3, wherein a molar ratio of cellulase:xylanase in the culture is greater than 4:1 or less than 1:4.

5. The culture of claim 1, wherein said specific activity ratio of cellulase:xylanase in the culture is greater than 10:1 or less than 1:10.

6. The culture of claim 5, wherein said specific activity ratio of cellulase:xylanase in the culture is less than HO.

7. The culture of claim 1, wherein an expression plasmid for expressing said secreted cellulase and said secreted xylanase is a pSIP-derived expression plasmid.

8. The culture of claim 1, wherein said biomass comprises lignocelluloses.

9. The culture of claim 1, wherein said biomass comprises cellulose and hemicellulose.

10. The culture of claim 9, wherein said biomass further comprises lignin.

11. The culture of claim 1, wherein said biomass is selected from the group consisting of paper, paper products, paper waste, wood, particle board, sawdust, agricultural waste, sewage, silage, grasses, rice hulls, bagasse, jute, hemp, flax, bamboo, sisal, abaca, straw, corn cobs, corn stover, switchgrass, alfalfa, hay, coconut hair, cotton, seaweed, algae, and mixtures thereof.

12. The population of lactic acid bacteria of claim 2, being Lactobacillus plantarum.

13. The culture of claim 1, wherein said first population of lactic acid bacteria comprises Lactobacillus plantarum.

14. The culture of claim 1, wherein said second population of lactic acid bacteria comprises Lactobacillus plantarum.

15. The culture claim 1, wherein said cellulase is a Thermobifida fusca cellulase.

16. The culture of claim 1, wherein said xylanase is a Thermobifida fusca xylanase.

17. The culture of claim 1, wherein said cellulase is a mesophilic bacteria cellulase.

18. The culture of claim 1, wherein said xylanase is a mesophilic bacteria xylanase.

19. The culture of claim 17, wherein said mesophilic bacteria is a Ruminococcus flavefaciens or Ruminococcus albus.

20. The culture of claim 1, wherein said cellulase comprises a leader sequence as set forth in SEQ ID NOs: 16 or 17.

21. The culture of claim 1, wherein said xylanase comprises a leader sequence as set forth in SEQ ID NOs: 16 or 17.

22. The culture of claim 1, wherein said first population and said second population comprise identical strains of bacteria.

23. The culture of claim 1, wherein said first population and said second population comprise non-identical strains of bacteria.

24. A method of processing lignocellulose comprising propagating the culture of claim 8, thereby processing the lignocellulose.

25. The method of claim 24, further comprising degrading the lignin of the lignocellulose.

26. Silage comprising the culture of claim 1.

27. (canceled)
